SEQUENCE LISTING (13 sequences)

SEQ ID NO: 1 - RNA, 15 nt, Artificial Sequence
  Description of Artificial Sequence  Nucleic Acid Substrate
nnnnnnuhnnnnnnn

SEQ ID NO: 2 - RNA, 36 nt, Artificial Sequence
  Description of Artificial Sequence  Enzymatic Nucleic Acid
nnnnnnncugaugagnnngaaannncgaaannnnnn

SEQ ID NO: 3 - RNA, 14 nt, Artificial Sequence
  Description of Artificial Sequence  Nucleic Acid Substrate
nnnnnchnnnnnnn

SEQ ID NO: 4 - RNA, 35 nt, Artificial Sequence
  Description of Artificial Sequence  Enzymatic Nucleic Acid
nnnnnnncugaugagnnngaaannncgaannnnnn

SEQ ID NO: 5 - RNA, 15 nt, Artificial Sequence
  Description of Artificial Sequence  Nucleic Acid Substrate
nnnnnnygnnnnnnn

SEQ ID NO: 6 - RNA, 35 nt, Artificial Sequence
  Description of Artificial Sequence  Enzymatic Nucleic Acid
nnnnnnnugauggcaugcacuaugcgcgnnnnnnn

SEQ ID NO: 7 - RNA, 48 nt, Artificial Sequence
  Description of Artificial Sequence  Enzymatic Nucleic Acid
gugugcaaccggaggaaacucccuucaaggacgaaaguccgggacggg

SEQ ID NO: 8 - RNA, 16 nt, Homo sapiens
gccguggguugcacac

SEQ ID NO: 9 - RNA, 36 nt, Artificial Sequence
  Description of Artificial Sequence  Enzymatic Nucleic Acid
gugccuggccgaaaggcgagugaggucugccgcgcn

SEQ ID NO: 10 - RNA, 15 nt, Homo sapiens
gcgcggcgcaggcac

SEQ ID NO: 11 - DNA, 16 bp, Artificial Sequence
  Description of Artificial Sequence  Enzymatic Nucleic Acid
rggctagctacaacga

SEQ ID NO: 12 - RNA, 10 nt, Artificial Sequence
  Description of Artificial Sequence  Enzymatic Nucleic Acid Stem Loop
gccguuaggc

SEQ ID NO: 13 - RNA, 35 nt, Artificial Sequence
  Description of Artificial Sequence  Enzymatic Nucleic Acid
gaguugcugaugaggccgaaaggccgaaagucugn

(19) United States
(12) Patent Application Publication
(10) Pub. No.: US 2003/0216335 A1
(43) Pub. Date: Nov. 20, 2003

(54) Method and reagent for the modulation of female reproductive diseases and conditions

(76) Inventors: Jennifer Lockridge, Westminster, CO (US); Pamela Pavco, Lafayette, CO (US); Gilad Gordon, Boulder, CO (US)

Correspondence Address: Anita J. Terpstra, Ph.D., McDonnell Boehnen Hulbert & Berghoff, 32nd Floor, 300 S. Wacker Drive, Chicago, IL, 60606, US

(21) Appl. No.: 10/306,747

(22) Filed: Nov. 27, 2002

Related U.S. Application Data
(60) Provisional application No. 60/334,461, filed on Nov. 30, 2001.

(51) Int. Cl. A61K 48/00

(52) U.S. Cl. 514/44

(57) ABSTRACT

The present invention relates to nucleic acid molecules, including dsRNA, siRNA, antisense, 2,5-A chimeras, aptamers, and enzymatic nucleic acid molecules, such as hammerhead ribozymes, DNAzymes, and allozymes, which modulate the expression of vascular endothelial growth factor receptor (VEGF) and/or vascular endothelial growth factor receptor (VEGFr) genes for the treatment and/or diagnosis of female reproductive disorders and conditions, including but not limited to endometriosis, endometrial carcinoma, gynecologic bleeding disorders, irregular menstrual cycles, ovulation, premenstrual syndrome (PMS), and menopausal dysfunction.

This application claims the benefit of U.S. Provisional Serial No. 60/334,461, filed Nov. 30, 2001.

### BACKGROUND OF THE INVENTION

This invention relates to methods and reagents for the treatment of diseases or conditions relating to the levels of expression of vascular endothelial growth factor (VEGF) and vascular endothelial growth factor receptor(s). Specifically, the instant invention features nucleic-acid based molecules and methods that modulate the expression of vascular endothelial growth factor and/or vascular endothelial growth factor receptors, such as VEGFR1 and/or VEGFR2, that are useful in treating, controlling and/or diagnosing female reproductive disorders and conditions, including but not limited to endometriosis, endometrial carcinoma, gynecologic bleeding disorders, irregular menstrual cycles, ovulation, premenstrual syndrome (PMS), and menopausal dysfunction.

The following is a discussion of relevant art, none of which is admitted to be prior art to the present invention.

The vascular endothelial growth factor (VEGF) family of angiogenic molecules is involved in both physiological angiogenesis, and a number of pathological conditions that are characterized by excessive angiogenesis. Increasing evidence suggests that the VEGF family may also be involved with both the etiology and maintenance of peritoneal endometriosis. Peritoneal endometriosis is a significant debilitating gynecological problem of widespread prevalence. It is now generally accepted that the pathogenesis of peritoneal endometriosis involves the implantation of exfoliated endometrium. Maintenance of exfoliated endometrial tissue is dependent upon the generation and maintenance of an extensive blood supply both within and surrounding the ectopic tissue.

Endometriosis is a disease affecting an estimated 77 million women and teenagers worldwide. Endometriosis is a leading cause of infertility, chronic pelvic pain and hysterectomy. Endometriosis can be characterized when endometrial tissue (the tissue inside the uterus which builds up and is shed each month during menses) is found outside the uterus, in other areas of the body. The endometrial tissue can respond to hormonal commands each month and break down and bleed. However, unlike the endometrium, these tissue deposits have no way of leaving the body. The result is internal bleeding, degeneration of blood and tissue shed from the growths, inflammation of the surrounding areas, expression of irritating enzymes and formation of scar tissue. In addition, depending on the location of the growths, interference with the bowel, bladder, intestines and other areas of the pelvic cavity can occur. Endometrial tissue has even been found lodged in the skin and at other extrapelvic locations like the arm, leg and even brain.

Currently, the presence of Endometriosis can only be confirmed through surgery such as laparoscopy, but can be suspected based on symptoms, physical findings and diagnostic tests. Endometriosis can be treated in many different ways, both surgically and medically. Most commonly, surgery will be performed during which the disease will be excised, ablated, fulgarated, cauterized or otherwise removed, and adhesions will also be freed. Surgeries include but are not limited to laparoscopy; laparotomy; presacral and uterosacral and various levels of hysterectomies, where some or all of the reproductive organs are removed. Often, this method will only relieve the symptoms associated with growths on the reproductive organs, not the bowels or kidneys and related areas where Endometriosis can be present.

There are several drugs used to treat Endometriosis that are utilized either alone or in combination with surgery. These include contraceptives, GnRH agonists, and/or synthetic hormones. GnRH agonists are commonly used on women in all stages of the disease and may sometimes have serious side affects. GNRH (gonadotropin releasing hormone) analogues are classified into 2 groups: agonists and antagonists. Agonists are commonly used in the treatment of Endometriosis by suppressing the manufacture of follicle stimulating hormone (FSH) and luteinizing hormone (LH), common hormones required in ovulation. When they are not secreted, the body will go into “pseudo-menopause,” stalling the growth of more implants. However, these are again only stop-gap measures that can be utilized only for short term intervals. Once the body returns to its normal state, the Endometriosis will again begin to implant itself.

Angiogenesis is likely to be involved in the pathogenesis of endometriosis. According to the transplantation theory, when the exfoliated endometrium is attached to the peritoneal layer, the establishment of a new blood supply is essential for the survival of the endometrial implant and development of endometriosis (Donnez et al., 1998, Hum. Reprod., 13, 1686-1690). Endometrial growth and repair after menstruation are associated with profound angiogenesis. Abnormalities in these processes result in excessive or unpredictable bleeding patterns and are common in many women. It is therefore important to understand which factors regulate normal endometrial angiogenesis. Vascular endothelial growth factor (VEGF) is an endothelial cell-specific mitogen that plays an important role in normal and pathological angiogenesis (Fasciani et al., 2000, Mol. Hum. Reprod., 6, 50-54; Sharkey et al., 2000, J. Clin. Endocrinol. Metab., 85, 402-409). Sources of this factor include the eutopic endometrium, ectopic endometriotic tissue and peritoneal fluid macrophages. Important to its etiology is the correct peritoneal environment in which the exfoliated endometrium is seeded and implants. Established ectopic tissue is then dependent on the peritoneal environment for its survival, an environment that supports angiogenesis. The increasing knowledge of the involvement of the VEGF family in endometriotic angiogenesis raises the possibility of novel approaches to its medical management, with particular focus on the anti-angiogenic control of the action of VEGF (McLaren, 2001, Hum. Reprod. Update, 6, 45-55).

VEGF, also referred to as vascular permeability factor (VPF) and vasculotropin, is a potent and highly specific mitogen of vascular endothelial cells (for a review see Ferrara, 1993 Trends Cardiovas. Med. 3, 244; Neufeld et al., 1994, Prog. Growth Factor Res. 5, 89). VEGF-induced neovascularization is implicated in various pathological conditions such as tumor angiogenesis, proliferative diabetic retinopathy, hypoxia-induced angiogenesis, rheumatoid arthritis, psoriasis, wound healing and others.

VEGF, an endothelial cell-specific mitogen, is a 34-45 kDa glycoprotein with a wide range of activities that include promotion of angiogenesis, enhancement of vascular-permeability and others. VEGF belongs to the platelet-derived growth factor (PDGF) family of growth factors with approximately 18% homology with the A and B chain of PDGF at the amino acid level. Additionally, VEGF contains the eight conserved cysteine residues common to all growth factors belonging to the PDGF family (Neufeld et al., supra). VEGF protein is believed to exist predominantly as disulfide-linked homodimers; monomers of VEGF have been shown to be inactive (Plouct et al., 1989 EMBO J. 8, 3801).

VEGF exerts its influence on vascular endothelial cells by binding to specific high-affinity cell surface receptors. Covalent cross-linking experiments with ¹²⁵I-labeled VEGF protein have led to the identification of three high molecular weight complexes of 225, 195 and 175 kDa presumed to be VEGF and VEGF receptor complexes (Vaisman et al., 1990 J. Biol. Chem. 265, 19461). Based on these studies VEGF-specific receptors of 180, 150 and 130 kDa molecular mass were predicted. In endothelial cells, receptors of 150 and 130 kDa have been identified. The VEGF receptors belong to the superfamily of receptor tyrosine kinases (RTKs) characterized by a conserved cytoplasmic catalytic kinase domain and a hydrophilic kinase sequence. The extracellular domains of the VEGF receptors consist of seven immunoglobulin-like domains that are thought to be involved in VEGF binding functions.

The two most abundant and high-affinity receptors of VEGF are flt-1 (VEGFR1) (fms-like tyrosine kinase) cloned by Shibuya et al., 1990 Oncogene 5, 519 and KDR (VEGFR2) (kinase-insert-domain-containing receptor) cloned by Terman et al., 1991 Oncogene 6, 1677. The murine homolog of KDR, cloned by Mathews et al., 1991, Proc. Natl. Acad. Sci., USA, 88, 9026, shares 85% amino acid homology with KDR and is termed as flk-1 (fetal liver kinase-1).

The high-affinity binding of VEGF to its receptors is modulated by cell surface-associated heparin and heparin-like molecules (Gitay-Goren et al., 1992 J. Biol. Chem. 267, 6093).

VEGF expression has been associated with several pathological states besides endometriosis, such as tumor angiogenesis, several forms of blindness, rheumatoid arthritis, psoriasis and others. In addition, a number of studies have demonstrated that VEGF is both necessary and sufficient for neovascularization. Takashita et al., 1995 J. Clin. Invest. 93, 662, demonstrated that a single injection of VEGF augmented collateral vessel development in a rabbit model of ischemia. VEGF also can induce neovascularization when injected into the cornea. Expression of the VEGF gene in CHO cells is sufficient to confer tumorigenic potential to the cells. Kim et al., supra and Millauer et al., supra used monoclonal antibodies against VEGF or a dominant negative form of VEGFR2 receptor to inhibit tumor-induced neovascularization.

During development, VEGF and its receptors are associated with regions of new vascular growth (Millauer et al., 1993 Cell 72, 835; Shalaby et al., 1993 J. Clin. Invest. 91, 2235). Furthermore, transgenic mice lacking either of the VEGF receptors are defective in blood vessel formation and these mice do not survive; VEGFR2 appears to be required for differentiation of endothelial cells, while VEGFR1 appears to be required at later stages of vessel formation (Shalaby et al., 1995 Nature 376, 62; Fung et al., 1995 Nature 376, 66). Thus, these receptors apparently need to be present to properly signal endothelial cells or their precursors to respond to vascularization-promoting stimuli.

Pavco et al., International PCT Publication No. WO 97/15662, describes methods and reagents for treating diseases or conditions related to levels of vascular endothelial growth factor receptor.

Robinson, International PCT Publication No. WO 95/04142, describes the use of certain antisense oligonucleotides targeted against VEGF RNA to inhibit VEGF expression.

Jellinek et al., 1994 Biochemistry 33, 10450 describe the use of specific VEGF-specific high-affinity RNA aptamers to inhibit the binding of VEGF to its receptors.

Rockwell and Goldstein, International PCT Publication No. WO 95/21868, describe the use of certain anti-VEGF receptor monoclonal antibodies to neutralize the effect of VEGF on endothelial cells.

Pappa, International PCT Publication No. WO 01/32920, describes inhibitors, including certain ribozyme and antisense nucleic acid molecules, of specific genes, including cathepsin D, AEBP-1, stromelysin-3, cystatin B, protease inhibitor 1, sFRP4, gelsolin, IGFBP-3, dual specificity phosphatase 1, PAEP, Ig gamma chain, ferritin, complement component 3, pro-alpha-1 type III collagen, proline 4-hydroxylase, alpha-2 type I collagen, claudin-4, melanoma adhesion protein, procollagen C-endopeptidase enhancer, nascent-polypeptide-associated complex alpha polypeptide, elongation factor 1 alpha (EF-1-alpha), vitamin D325 hydroxylase, CSRP-1, steroidogenic acute regulatory protein, apolipoprotein E, transcobalamin II, prosaposin, early growth response 1 (EGRI), ribosomal protein S6, adenosine deaminase RNA-specific protein, RAD21, guanine nucleotide binding protein beta polypeptide 2-like 1 (RACKI) and podocalyxin genes which are all differentially expressed in tissues within individual patients with endometriosis.

Labarbera et al., International PCT Publication No. WO 00/73416, describes specific antisense nucleic acid molecules targeting follicle-stimulating hormone receptor.

Storella et al., International PCT Publication No. WO 99/63116, describes modulators of Prothymosin gene products for treating endometriosis, including certain ribozymes and antisense nucleic acid molecules.

### SUMMARY OF THE INVENTION

This invention features nucleic acid-based molecules, for example, enzymatic nucleic acid molecules, allozymes, antisense nucleic acids, 2-5A antisense chimeras, triplex forming oligonucleotides, decoy RNA, dsRNA, siRNA, aptamers, and antisense nucleic acids containing nucleic acid cleaving chemical groups, and methods to modulate vascular endothelial growth factor (VEGF) and/or vascular endothelial growth factor receptor (VEGFr) gene expression. Non-limiting examples of genes that encode vascular endothelial growth factor receptors of the invention include VEGFR1, VEGFR2 or combinations thereof. In particular, the instant invention features nucleic acid-based molecules and methods that modulate the expression of vascular endothelial growth factor and/or vascular endothelial growth factor receptors, such as VEGFR1 and/or VEGFR2, that are useful in treating, controlling, and/or diagnosing female reproductive disorders and conditions, including but not limited to endometriosis, endometrial carcinoma, gynecologic bleeding disorders, irregular menstrual cycles, ovulation, premenstrual syndrome (PMS), and menopausal dysfunction.

In one embodiment, the invention features one or more nucleic acid-based molecules and methods that independently or in combination modulate the expression of gene(s) encoding vascular endothelial growth factor receptors. Specifically, the present invention features nucleic acid molecules that modulate the expression of VEGF (for example Genbank Accession No.

NM_{—}003376), VEGFR1 receptor (for example Genbank Accession No. NM_{—}002019), and VEGFR2 receptor (for example Genbank Accession No. NM_{—}002253) that are useful in treating, controlling, and/or diagnosing female reproductive disorders and conditions, including but not limited to endometriosis, endometrial carcinoma, gynecologic bleeding disorders, irregular menstrual cycles, ovulation, premenstrual syndrome (PMS), and menopausal dysfunction.

In another embodiment, the present invention features a compound having Formula I: (SEQ ID NO: 13)

5′ gₛaₛgₛuₛugcUGAuGagg ccgaaa ggccGaaAgucugB 3′

wherein each a is 2′-O-methyl adenosine nucleotide, each g is a 2′-O-methyl guanosine nucleotide, each c is a 2′-O-methyl cytidine nucleotide, each u is a 2′-O-methyl uridine nucleotide, each A is adenosine, each G is guanosine, each s individually represents a phosphorothioate internucleotide linkage, U is 2′-deoxy-2′-C-allyl uridine, and B is an inverted deoxyabasic moiety. This compound is also referred to as ANGIOZYMETM ribozyme.

In one embodiment, the invention features a composition comprising a nucleic acid molecule of the invention in an acceptable carrier. In another embodiment, the invention features a pharmaceutical composition comprising a compound of Formula I in a pharmaceutically acceptable carrier. In one embodiment, the invention features a method of administering to a cell, for example a mammalian cell or human cell, a nucleic acid molecule of the inventioncomprising contacting the cell with the nucleic acid molecule under conditions suitable for administration, for example, in the presence of a delivery reagent such as a lipid, cationic lipid, phospholipid, or liposome. In another embodiment, the invention features a method of administering to a cell, for example a mammalian cell or human cell, a compound of Formula I comprising contacting the cell with the compound under conditions suitable for administration, for example, in the presence of a delivery reagent such as a lipid, cationic lipid, phospholipid, or liposome.

In one embodiment, the present invention features a mammalian cell comprising a nucleic acid molecule of the invention, wherein the mammalian cell is, for example, a human cell. In another embodiment, the present invention also- features a mammalian cell comprising the compound of Formula I, wherein the mammalian cell is, for example, a human cell.

In one embodiment, the invention features a method of inhibiting angiogenesis, for example endometrial neovascularization, in a subject comprising contacting the subject with a nucleic acid molecule of the invention, under conditions suitable for the inhibition. In another embodiment, the invention features a method of inhibiting angiogenesis, for example endometrial neovascularization, in a subject comprising contacting the subject with a compound of Formula I under conditions suitable for the inhibition.

In another embodiment, the invention features a method of treatment of a subject having a condition associated with an increased level of VEGR and/or a VEGF receptor, for example endometriosis, endometrial carcinoma, gynecologic bleeding disorders, irregular menstrual cycles, ovulation, premenstrual syndrome (PMS), or menopausal dysfunction, comprising contacting cells of the patient with a nucleic acid molecule of the invention, such as a compound of Formula I, under conditions suitable for the treatment.

In yet another embodiment, a method of treatment of the invention further comprises the use of one or more drug therapies under conditions suitable for the treatment. Non-limiting examples of other drug therapies that can be used in combination with nucleic acid molecules of the invention include GnRH (gonadotropin releasing hormone) agonists, Lupron Depot (Leuprolide Acetate), Synarel (naferalin acetate), Zolodex (goserelin acetate), Suprefact (buserelin acetate), Danazol, or oral contraceptives including but not limited to Depo-Provera or Provera (medroxyprogesterone acetate), or any other estrogen/progesterone contraceptive.

In one embodiment, the invention features a method of administering to a mammalian subject, for example a human, a nucleic acid molecule of the invention comprising contacting the mammalian subject with the nucleic acid molecule under conditions suitable for the administration, for example, in the presence of a delivery reagent such as a lipid, cationic lipid, phospholipid, or liposome. In another embodiment, the invention features a method of administering to a mammalian subject, for example a human, a compound of Formula I comprising contacting the mammalian subject with the compound under conditions suitable for the administration, for example, in the presence of a delivery reagent such as a lipid, cationic lipid, phospholipid, or liposome.

In one embodiment, the invention features a nucleic acid molecule which down regulates expression of a vascular endothelial growth factor (VEGF) and/or vascular endothelial growth factor receptor (VEGFr) gene, for example, wherein the VEGFr gene comprises VEGFR1 or VEGFR2 and any combination thereof.

In one embodiment, a nucleic acid molecule, such as an enzymatic nucleic acid molecule, antisense nucleic acid molecule, 2-5A antisense chimera, triplex forming oligonucleotide, decoy RNA, dsRNA, siRNA, aptamer, or antisense nucleic acid containing nucleic acid cleaving chemical groups of the invention is adapted to treat or control endometriosis, endometrial carcinoma, gynecologic bleeding disorders, irregular menstrual cycles, ovulation, premenstrual syndrome (PMS), or menopausal dysfunction.

In another embodiment, an enzymatic nucleic acid molecule, antisense nucleic acid molecule, 2-5A antisense chimera, triplex forming oligonucleotide, decoy RNA, dsRNA, siRNA, aptamer, or antisense nucleic acid containing nucleic acid cleaving chemical groups of the invention is adapted for birth control.

In one embodiment, an enzymatic nucleic acid molecule of the invention is in a hammerhead, Inozyme, Zinzyme, DNAzyme, Amberzyme, or G-cleaver configuration.

In one embodiment, an enzymatic nucleic acid molecule of the invention comprises between 8 and 100 bases complementary to RNA of VEGFR1 and/or VEGFR2 gene. In another embodiment, an enzymatic nucleic acid molecule of the invention comprises between 14 and 24 bases complementary to RNA of VEGFR1 and/or VEGFR2 gene.

In one embodiment, a siRNA molecule of the invention comprises a double stranded RNA wherein one strand of the RNA is complementary to RNA of a VEGFR1 and/or VEGFR2 gene. In another embodiment, a siRNA molecule of the invention comprises a double stranded RNA wherein one strand of the RNA comprises a portion of a sequence of RNA having a VEGFR1 and/or VEGFR2 sequence. In yet another embodiment, a siRNA molecule of the invention comprises a double stranded RNA wherein both strands of RNA are connected by a non-nucleotide linker. Alternately, a siRNA molecule of the invention comprises a double stranded RNA wherein both strands of RNA are connected by a nucleotide linker, such as a loop or stem loop structure.

In one embodiment, a single strand component of a siRNA molecule of the invention is from about 14 to about 50 nucleotides in length. In another embodiment, a single strand component of a siRNA molecule of the invention is about 14, 15, 16, 17, 18, 19, 20, 21, 22, 23, 24, 25, 26, 27, or 28 nucleotides in length. In yet another embodiment, a single strand component of a siRNA molecule of the invention is about 23 nucleotides in length. In one embodiment, a siRNA molecule of the invention is from about 28 to about 56 nucleotides in length. In another embodiment, a siRNA molecule of the invention is about 40, 41, 42, 43, 44, 45, 46, 47, 48, 49, 50, 51, or 52 nucleotides in length. In yet another embodiment, a siRNA molecule of the invention is about 46 nucleotides in length.

In one embodiment, an enzymatic nucleic acid molecule, antisense nucleic acid molecule, 2-SA antisense chimera, triplex forming oligonucleotide, decoy RNA, dsRNA, siRNA, aptamer, or antisense nucleic acid containing nucleic acid cleaving chemical groups of the invention is chemically synthesized.

In another embodiment, an enzymatic nucleic acid molecule, antisense nucleic acid molecule, 2-SA antisense chimera, triplex forming oligonucleotide, decoy RNA, dsRNA, siRNA, aptamer, or antisense nucleic acid containing nucleic acid cleaving chemical groups of the invention comprises at least one 2′-sugar modification.

In another embodiment, an enzymatic nucleic acid molecule, antisense nucleic acid molecule, 2-SA antisense chimera, triplex forming oligonucleotide, decoy RNA, dsRNA, siRNA, aptamer, or antisense nucleic acids containing nucleic acid cleaving chemical groups of the invention comprises at least one nucleic acid base modification.

In another embodiment, an enzymatic nucleic acid molecule, antisense nucleic acid molecule, 2-SA antisense chimera, triplex forming oligonucleotide, decoy RNA, dsRNA, siRNA, aptamer, or antisense nucleic acid containing nucleic acid cleaving chemical groups of the invention comprises at least one phosphate backbone modification.

In one embodiment, the invention features a mammalian cell, for example a human cell, including a nucleic acid molecule of the invention.

In another embodiment, the invention features a method of reducing VEGF and/or VEGFr, such as VEGFR1 and/or VEGFR2, expression or activity in a cell comprising contacting the cell with a nucleic acid molecule of the invention that modulates the expression and/or activity of VEGF and/or VEGFr under conditions suitable for the reduction.

In another embodiment, a method of treatment of a patient having a condition associated with the level of VEGF and/or VEGFr, such as VEGFR1 and/or VEGFR2 is featured, wherein the method further comprises the use of one or more drug therapies under conditions suitable for the treatment.

In one embodiment, the invention features a method for treatment of a subject having endometriosis, endometrial carcinoma, gynecologic bleeding disorders, irregular menstrual cycles, ovulation, premenstrual syndrome (PMS), or menopausal dysfunction, comprising administering to the subject a nucleic acid molecule of the invention that modulates the expression and/or activity of VEGF and/or VEGFr under conditions suitable for the treatment.

In another embodiment, the invention features a method for birth control in a subject comprising administering to the subject a nucleic acid molecule of the invention that modulates the expression and/or activity of VEGF and/or VEGFr under conditions suitable for the treatment.

In another embodiment, the invention features a method of cleaving RNA encoded by a VEGF, VEGFR1 and/or VEGFR2 gene comprising contacting an enzymatic nucleic acid molecule of the invention having endonuclease activity with RNA encoded by a VEGFR1 and/or VEGFR2 gene under conditions suitable for the cleavage, for example, wherein the cleavage is carried out in the presence of a divalent cation, such as Mg²⁺.

In one embodiment, a nucleic acid molecule of the invention comprises a cap structure, for example a 3′,3′-linked or 5′,5′-linked deoxyabasic ribose derivative, wherein the cap structure is at the 5′-end, or 3′-end, or both the 5′-end and the 3′-end of the enzymatic nucleic acid molecule.

In another embodiment, a nucleic acid molecule of the invention comprises a cap structure, for example a 3′,3′-linked or 5′,5′-linked deoxyabasic ribose derivative, wherein the cap structure is at the 5′-end, or 3′-end, or both the 5′-end and the 3′-end of the antisense nucleic acid molecule.

In another embodiment, a nucleic acid molecule of the invention comprises a cap structure, for example a 3′,3′-linked or 5′,5′-linked deoxyabasic ribose derivative, wherein the cap structure is at the 5′-end, or 3′-end, or both the 5′-end and the 3′-end of the siRNA molecule.

In one embodiment, the invention features an expression vector comprising a nucleic acid sequence encoding at least one nucleic acid molecule of the invention, such that the vector allows expression of the nucleic acid molecule.

In another embodiment, the invention features a mammalian cell, for example, a human cell, comprising an expression vector of the invention.

In yet another embodiment, an expression vector of the invention further comprises a sequence for a nucleic acid molecule complementary to RNA encoded by a VEGF and/or VEGFr, such as VEGFR1 and/or VEGFR2 gene.

In one embodiment, an expression vector of the invention comprises a nucleic acid sequence encoding two or more nucleic acid molecules of the invention, which can be the same or different.

In another embodiment, the invention features a method for treatment or control of endometriosis, endometrial carcinoma, gynecologic bleeding disorders, irregular menstrual cycles, ovulation, premenstrual syndrome (PMS), or menopausal dysfunction, comprising administering to a patient a nucleic acid molecule of the invention that modulates the expression and/or activity of VEGF and/or VEGFr, such as an enzymatic nucleic acid molecule, antisense nucleic acid molecule, 2-5A antisense chimera, triplex forming oligonucleotide, decoy RNA, dsRNA, siRNA, aptamer, or antisense nucleic acid containing nucleic acid cleaving chemical groups of the invention, under conditions suitable for the treatment, including administering to the patient one or more other therapies, for example, GnRH (gonadotropin releasing hormone) agonists, Lupron Depot (Leuprolide Acetate), Synarel (naferalin acetate), Zolodex (goserelin acetate), Suprefact (buserelin acetate), Danazol, or oral contraceptives including but not limited to Depo-Provera or Provera (medroxyprogesterone acetate), or any other estrogen/progesterone contraceptive.

In one embodiment, the method of treatment features a nucleic acid molecule of the invention, such as an enzymatic nucleic acid, antisense nucleic acid molecule or siRNA molecule, that comprises at least five ribose residues, at least ten 2′-O-methyl modifications, and a 3′- end modification, such as a 3′-3′ inverted a basic moiety. In another embodiment, a nucleic acid molecule of the invention further comprises phosphorothioate linkages on at least three of the 5′ terminal nucleotides.

In another embodiment, the invention features a method of administering to a mammal, for example a human, an enzymatic nucleic acid molecule, antisense nucleic acid molecule, 2-5A antisense chimera, triplex forming oligonucleotide, decoy RNA, dsRNA, siRNA, aptamer, or antisense nucleic acid containing nucleic acid cleaving chemical groups of the invention, comprising contacting the mammal with the nucleic acid molecule under conditions suitable for the administration, for example, in the presence of a delivery reagent such as a lipid, cationic lipid, phospholipid, or liposome.

In yet another embodiment, the invention features a method of administering to a mammal an enzymatic nucleic acid molecule, antisense nucleic acid molecule, 2-5A antisense chimera, triplex forming oligonucleotide, decoy RNA, dsRNA, siRNA, aptamer, or antisense nucleic acid containing nucleic acid cleaving chemical groups of the invention in conjunction with other therapies, comprising contacting the mammal, for example a human, with the nucleic acid molecule and the other therapy under conditions suitable for the administration.

In another embodiment, other therapies contemplated by the instant invention that can be used in conjunction with the nucleic acid molecules of the instant invention include, but are not limited to GnRH (gonadotropin releasing hormone) agonists, Lupron Depot (Leuprolide Acetate), Synarel (naferalin acetate), Zolodex (goserelin acetate), Suprefact (buserelin acetate), Danazol, or oral contraceptives including but not limited to Depo-Provera or Provera (medroxyprogesterone acetate), or any other estrogen/progesterone contraceptive.

In one embodiment, the invention features the use of an enzymatic nucleic acid molecule, preferably in the hammerhead, NCH, G-cleaver, Amberzyme, Zinzyme, and/or DNAzyme motif, to down-regulate the expression of VEGFR1 and/or VEGFR2 genes in the treatment or control of endometriosis, endometrial carcinoma, gynecologic bleeding disorders, irregular menstrual cycles, ovulation, premenstrual syndrome (PMS), or menopausal dysfunction.

In another embodiment, the invention features the use of an enzymatic nucleic acid molecule, preferably in the hammerhead, NCH, G-cleaver, Amberzyme, Zinzyme, and/or DNAzyme motif, to down-regulate the expression of VEGF and/or VEGFr, such as VEGFR1 and/or VEGFR2 genes as a method of birth control.

By “inhibit”, “down-regulate”, or “reduce”, it is meant that the expression of the gene, or level of nucleic acids or equivalent nucleic acids encoding one or more proteins or protein subunits, or activity of one or more proteins or protein subunits, such as VEGFR1 and/or flk-1, is reduced below that observed in the absence of the nucleic acid molecules of the invention. In one embodiment, inhibition, down-regulation or reduction with an enzymatic nucleic acid molecule preferably is below that level observed in the presence of an enzymatically inactive or attenuated molecule that is able to bind to the same site on the target nucleic acid, but is unable to cleave that nucleic acid. In another embodiment, inhibition, down-regulation, or reduction with antisense oligonucleotides is preferably below that level observed in the presence of, for example, an oligonucleotide with scrambled sequence or with mismatches. In another embodiment, inhibition, down-regulation, or reduction of VEGF and/or VEGFr, such as VEGFR1 and/or VEGFR2, with the nucleic acid molecule of the instant invention is greater in the presence of the nucleic acid molecule than in its absence.

By “up-regulate” is meant that the expression of a gene, or level of nucleic acids or equivalent nucleic acids encoding one or more proteins or protein subunits, or activity of one or more proteins or protein subunits, such as VEGFR1 and/or VEGFR2, is greater than that observed in the absence of the nucleic acid molecules of the invention. For example, the expression of a gene, such as VEGF and/or VEGFr, such as VEGFR1 and/or VEGFR2 gene, can be increased in order to treat, prevent, ameliorate, or modulate a pathological condition caused or exacerbated by an absence or low level of gene expression.

By “modulate” is meant that the expression of a gene, or level of nucleic acids or equivalent nucleic acids encoding one or more proteins or protein subunits, or activity of one or more proteins protein subunit(s) is up-regulated or down-regulated, such that the expression, level, or activity is greater than or less than that observed in the absence of the nucleic acid molecules of the invention.

By “enzymatic nucleic acid molecule” it is meant a nucleic acid molecule which has complementarity in a substrate binding region to a specified gene target, and also has an enzymatic activity which is active to specifically cleave a target nucleic acid. That is, the enzymatic nucleic acid molecule is able to intermolecularly cleave a nucleic acid and thereby inactivate a target nucleic acid molecule. These complementary regions allow sufficient hybridization of the enzymatic nucleic acid molecule to the target nucleic acid and thus permit cleavage. One hundred percent complementarity is preferred, but complementarity as low as 50-75% can also be useful in this invention (see for example Werner and Uhlenbeck, 1995, Nucleic Acids Research, 23, 2092-2096; Hammann et al., 1999, Antisense and Nucleic Acid Drug Dev., 9, 25-31). The nucleic acids can be modified at the base, sugar, and/or phosphate groups. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not limiting in the invention and those skilled in the art will recognize that all that is important in an enzymatic nucleic acid molecule of this invention is that it has a specific substrate binding site which is complementary to one or more of the target nucleic acid regions, and that it have nucleotide sequences within or surrounding that substrate binding site which impart a nucleic acid cleaving and/or ligation activity to the molecule (Cech et al., U.S. Pat. No. 4,987,071; Cech et al., 1988, 260 JAMA 3030).

Several varieties of naturally-occurring enzymatic nucleic acids are known presently. Each can catalyze the hydrolysis of nucleic acid phosphodiester bonds in trans (and thus can cleave other nucleic acid molecules) under physiological conditions. Table 1 summarizes some of the characteristics of these ribozymes. In general, enzymatic nucleic acids act by first binding to a target nucleic acid. Such binding occurs through the target binding portion of a enzymatic nucleic acid which is held in close proximity to an enzymatic portion of the molecule that acts to cleave the target nucleic acid. Thus, the enzymatic nucleic acid first recognizes and then binds a target nucleic acid through complementary base-pairing, and once bound to the correct site, acts enzymatically to cut the target nucleic acid. Strategic cleavage of such a target nucleic acid will destroy its ability to direct synthesis of an encoded protein. After an enzymatic nucleic acid has bound and cleaved its nucleic acid target, it is released from that nucleic acid to search for another target and can repeatedly bind and cleave new targets. Thus, a single ribozyme molecule is able to cleave many molecules of target nucleic acid. In addition, the ribozyme is a highly specific inhibitor of gene expression, with the specificity of inhibition depending not only on the base-pairing mechanism of binding to the target nucleic acid, but also on the mechanism of target nucleic acid cleavage. Single mismatches, or base-substitutions, near the site of cleavage can completely eliminate catalytic activity of a ribozyme.

In one embodiment of the inventions described herein, an enzymatic nucleic acid molecule of the invention is formed in a hammerhead or hairpin motif, but can also be formed in the motif of a hepatitis delta virus, group I intron, group II intron or RNase P RNA (in association with an RNA guide sequence), Neurospora VS RNA, DNAzymes, NCH cleaving motifs, or G-cleavers. Examples of such hammerhead motifs are described by Dreyfus, supra, Rossi et al., 1992, AIDS Research and Human Retroviruses 8, 183; of hairpin motifs by Hampel et al., EP0360257, Hampel and Tritz, 1989 Biochemistry 28, 4929, Feldstein et al., 1989, Gene 82, 53, Haseloff and Gerlach, 1989, Gene, 82, 43, and Hampel et al., 1990 Nucleic Acids Res. 18, 299; Chowrira & McSwiggen, U.S. Pat. No. 5,631,359; an examples of a hepatitis delta virus motif is described by Perrotta and Been, 1992 Biochemistry 31, 16; examples of RNase P motifs are described by Guerrier-Takada et al., 1983 Cell 35, 849; Forster and Altman, 1990, Science 249, 783; Li and Altman, 1996, Nucleic Acids Res. 24, 835; examples of Neurospora VS RNA ribozyme motifs are described by Collins (Saville and Collins, 1990 Cell 61, 685-696; Saville and Collins, 1991 Proc. Natl. Acad. Sci. USA 88, 8826-8830; Collins and Olive, 1993 Biochemistry 32, 2795-2799; Guo and Collins, 1995, EMBO. J 14, 363); examples of Group II introns are described by Griffin et al., 1995, Chem. Biol. 2, 761; Michels and Pyle, 1995, Biochemistry 34, 2965; Pyle et al., International PCT Publication No. WO 96/22689; an example of a Group I intron is described by Cech et al., U.S. Pat. No. 4,987,071; and examples of DNAzymes are described by Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262, and Beigelman et al., International PCT publication No. WO 99/55857. NCH cleaving motifs are described in Ludwig & Sproat, International PCT Publication No. WO 98/58058; and G-cleavers are described in Kore et al., 1998, Nucleic Acids Research 26, 4116-4120 and Eckstein et al., International PCT Publication No. WO 99/16871. Additional motifs such as the Aptazyme (Breaker et al, WO 98/43993), Amberzyme (FIG. 3; Beigelman et al., U.S. Pat. No. 6,482,932) and Zinzyme (FIG. 4) (Beigelman et al, U.S. Ser. No. 09/918,728), all included by reference herein including drawings, can also be used in the present invention. These specific motifs or configurations are not limiting in the invention and those skilled in the art will recognize that all that is important in an enzymatic nucleic acid molecule of this invention is that it have a specific substrate binding site which is complementary to one or more of the target gene RNA regions, and that it have nucleotide sequences within or surrounding that substrate binding site which impart a RNA cleaving activity to the molecule (Cech et al., U.S. Pat. No. 4,987,071).

By “nucleic acid molecule” as used herein is meant a molecule having nucleotides. The nucleic acid can be single, double, or multiple stranded and can comprise modified or unmodified nucleotides or non-nucleotides or various mixtures and combinations thereof.

By “enzymatic portion” or “catalytic domain” is meant that portion/region of a enzymatic nucleic acid molecule essential for cleavage of a nucleic acid substrate (for example see FIG. 1).

By “substrate binding arm” or “substrate binding domain” is meant that portion/region of a enzymatic nucleic acid which is able to interact, for example via complementarity (i.e., able to base-pair with), with a portion of its substrate. Preferably, such complementarity is 100%, but can be less if desired. For example, as few as 10 bases out of 14 can be base-paired (see for example Werner and Uhlenbeck, 1995, Nucleic Acids Research, 23, 2092-2096; Hammann et al., 1999, Antisense and Nucleic Acid Drug Dev., 9, 25-31). Examples of such arms are shown generally in FIGS. 1-4. That is, these arms contain sequences within a enzymatic nucleic acid which are intended to bring enzymatic nucleic acid and target nucleic acid together through complementary base-pairing interactions. An enzymatic nucleic acid of the invention can have binding arms that are contiguous or non-contiguous and can be of varying lengths. The length of the binding arm(s) are preferably greater than or equal to four nucleotides and of sufficient length to stably interact with the target nucleic acid; preferably 12-100 nucleotides; more preferably 14-24 nucleotides long (see for example Werner and Uhlenbeck, supra; Hamman et al., supra; Hampel et al., EP0360257; Berzal-Herranz et al., 1993, EMBO J, 12, 2567-73) or between 8 and 14 nucleotides long. If two binding arms are chosen, the design is such that the length of the binding arms are symmetrical (i.e., each of the binding arms is of the same length; e.g., four and four, five and five nucleotides, or six and six nucleotides, or seven and seven nucleotides long) or asymmetrical (i.e., the binding arms are of different length; e.g., three and five, six and three nucleotides; three and six nucleotides long; four and five nucleotides long; four and six nucleotides long; four and seven nucleotides long; and the like).

By “Inozyme” or “NCH” motif or configuration is meant, an enzymatic nucleic acid molecule comprising a motif as is generally described as NCH Rz in FIG. 2 and in Ludwig et al., International PCT Publication No. WO 98/58058 and U.S. patent application Ser. No. 08/878,640. Inozymes possess endonuclease activity to cleave nucleic acid substrates having a cleavage triplet NCH/, where N is a nucleotide, C is cytidine and H is adenosine, uridine or cytidine, and “/” represents the cleavage site. H is used interchangeably with X. Inozymes can also possess endonuclease activity to cleave nucleic acid substrates having a cleavage triplet NCN/, where N is a nucleotide, C is cytidine, and “/” represents the cleavage site. “I” in FIG. 2 represents an Inosine nucleotide, preferably a ribo-Inosine or xylo-Inosine nucleoside.

By “G-cleaver” motif or configuration is meant, an enzymatic nucleic acid molecule comprising a motif as is generally described as G-cleaver Rz in FIG. 2 and in Eckstein et al., U.S. Pat. No. 6,127,173. G-cleavers possess endonuclease activity to cleave nucleic acid substrates having a cleavage triplet NYN/, where N is a nucleotide, Y is uridine or cytidine and “/” represents the cleavage site. G-cleavers can be chemically modified as is generally shown in FIG. 2.

By “amberzyme” motif or configuration is meant, an enzymatic nucleic acid molecule comprising a motif as is generally described in FIG. 3 and in Beigelman et al., International PCT publication No. WO 99/55857 and U.S. patent application Ser. No. 09/476,387.

Amberzymes possess endonuclease activity to cleave nucleic acid substrates having a cleavage triplet NG/N, where N is a nucleotide, G is guanosine, and “/” represents the cleavage site.

Amberzymes can be chemically modified to increase nuclease stability through substitutions as are generally shown in FIG. 3. In addition, differing nucleoside and/or non-nucleoside linkers can be used to substitute the 5′-gaaa-3′ loops shown in the figure. Amberzymes represent a non-limiting example of an enzymatic nucleic acid molecule that does not require a ribonucleotide (2′-OH) group within its own nucleic acid sequence for activity.

By “zinzyme” motif or configuration is meant, an enzymatic nucleic acid molecule comprising a motif as is generally described in FIG. 4 and in Beigelman et al., International PCT publication No. WO 99/55857 and U.S. patent application Ser. No. 09/918,728. Zinzymes possess endonuclease activity to cleave nucleic acid substrates having a cleavage triplet including but not limited to YG/Y, where Y is uridine or cytidine, and G is guanosine and “/” represents the cleavage site. Zinzymes can be chemically modified to increase nuclease stability through substitutions as are generally shown in FIG. 4, including substituting 2′-O-methyl guanosine nucleotides for guanosine nucleotides. In addition, differing nucleotide and/or non-nucleotide linkers can be used to substitute the 5′-gaaa-2′ loop shown in the figure. Zinzymes represent a non-limiting example of an enzymatic nucleic acid molecule that does not require a ribonucleotide (2′-OH) group within its own nucleic acid sequence for activity.

By ‘DNAzyme’ is meant, an enzymatic nucleic acid molecule that does not require the presence of a 2′-OH group within its own nucleic acid sequence for activity. In particular embodiments the enzymatic nucleic acid molecule can have an attached linker or linkers, or other attached or associated groups, moieties, or chains containing one or more nucleotides with 2′-OH groups. DNAzymes can be synthesized chemically or expressed endogenously in vivo, by means of a single stranded DNA vector or equivalent thereof. An example of a DNAzyme is shown in FIG. 5 and is generally reviewed in Usman et al., U.S. Pat. No., 6,159,714; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422-423; and Santoro et. al., 2000, J. Am. Chem. Soc., 122, 2433-39. The “10-23” DNAzyme motif is one particular type of DNAzyme that was evolved using in vitro selection, see Santoro et al., supra and as generally described in Joyce et al., U.S. Pat. No. 5,807,718. Additional DNAzyme motifs can be selected for using techniques similar to those described in these references, and hence, are within the scope of the present invention.

By “sufficient length” is meant a nucleic acid molecule of the invention is long enough to provide the intended function under the expected condition. For example, a nucleic acid molecule of the invention needs to be of “sufficient length” to provide stable interaction with a target nucleic acid molecule under the expected binding conditions and environment. In another non-limiting example, for the binding arms of an enzymatic nucleic acid, “sufficient length” means that the binding arm sequence is long enough to provide stable binding to a target site under the expected reaction conditions and environment. The binding arms are not so long as to prevent useful turnover of the nucleic acid molecule.

By “stably interact” is meant interaction of an oligonucleotides with target nucleic acid (e.g., by forming hydrogen bonds with complementary nucleotides in the target under physiological conditions) that is sufficient to the intended purpose (e.g., cleavage of target nucleic acid by an enzyme). By “equivalent” RNA to VEGF, VEGFR1 and/or VEGFR2 is meant to include nucleic acid molecules having homology (partial or complete) to a nucleic acid encoding VEGF, VEGFR1 and/or VEGFR2 proteins or encoding proteins with similar function as VEGF, VEGFR1 and/or VEGFR2 proteins in various organisms, including human, rodent, primate, rabbit, pig, protozoans, fungi, plants, and other microorganisms and parasites. The equivalent nucleic acid sequence also includes, in addition to the coding region, regions such as 5′-untranslated region, 3′-untranslated region, introns, intron-exon junction and the like.

By “homology” is meant the nucleotide sequence of two or more nucleic acid molecules is partially or completely identical.

By “antisense nucleic acid”, it is meant a non-enzymatic nucleic acid molecule that binds to target nucleic acid by means of RNA-RNA or RNA-DNA or RNA-PNA (protein nucleic acid;

Egholm et al., 1993 Nature 365, 566) interactions and alters the activity of the target nucleic acid (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, an antisense molecule can be complementary to two (or even more) non-contiguous substrate sequences or two (or even more) non-contiguous sequence portions of an antisense molecule can be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49. In addition, antisense DNA can be used to target nucleic acid by means of DNA-RNA interactions, thereby activating RNase H, which digests the target nucleic acid in the duplex. The antisense oligonucleotides can comprise one or more RNAse H activating region, which is capable of activating RNAse H cleavage of a target nucleic acid. Antisense DNA can be synthesized chemically or expressed via the use of a single stranded DNA expression vector or equivalent thereof.

By “RNase H activating region” is meant a region (generally greater than or equal to 4-25 nucleotides in length, preferably from 5-11 nucleotides in length) of a nucleic acid molecule capable of binding to a target nucleic acid to form a non-covalent complex that is recognized by cellular RNase H enzyme (see for example Arrow et al., U.S. Pat. No. 5,849,902; Arrow et al., U.S. Pat. No. 5,989,912). The RNase H enzyme binds to a nucleic acid molecule-target nucleic acid complex and cleaves the target nucleic acid sequence. The RNase H activating region comprises, for example, phosphodiester, phosphorothioate (preferably at least four of the nucleotides are phosphorothiote substitutions; more specifically, 4-11 of the nucleotides are phosphorothiote substitutions); phosphorodithioate, 5′-thiophosphate, or methylphosphonate backbone chemistry or a combination thereof. In addition to one or more backbone chemistries described above, the RNase H activating region can also comprise a variety of sugar chemistries.

For example, the RNase H activating region can comprise deoxyribose, arabino, fluoroarabino or a combination thereof, nucleotide sugar chemistry. Those skilled in the art will recognize that the foregoing are non-limiting examples and that any combination of phosphate, sugar and base chemistry of a nucleic acid that supports the activity of RNase H enzyme is within the scope of the definition of the RNase H activating region and the instant invention.

By “2-5A antisense chimera” is meant an antisense oligonucleotide containing a 5′-phosphorylated 2′-5′-linked adenylate residue. These chimeras bind to target nucleic acid in a sequence-specific manner and activate a cellular 2-5A-dependent ribonuclease which, in turn, cleaves the target nucleic acid (Torrence et al, 1993 Proc. Natl. Acad. Sci. USA 90, 1300; Silverman et al., 2000, Methods Enzymol., 313, 522-533; Player and Torrence, 1998, Pharmacol. Ther., 78, 55-113).

By “triplex forming oligonucleotides” is meant an oligonucleotide that can bind to a double-stranded polynucleotide, such as DNA, in a sequence-specific manner to form a triple-strand helix. Formation of such triple helix structure has been shown to inhibit transcription of the targeted gene (Duval-Valentin et al., 1992 Proc. Natl. Acad. Sci. USA 89, 504; Fox, 2000, Curr. Med. Chem., 7, 17-37; Praseuth et. al., 2000, Biochim. Biophys. Acta, 1489, 181-206).

By “gene” it is meant a nucleic acid that encodes an RNA, for example, nucleic acid sequences including but not limited to structural genes encoding a polypeptide.

The term “complementarity” as used herein refers to the ability of a nucleic acid to form hydrogen bond(s) with another nucleic acid sequence by either traditional Watson-Crick or other non-traditional types. In reference to nucleic molecules of the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “RNA” is meant a molecule comprising at least one ribonucleotide residue. By “ribonucleotide” or “2′-OH” is meant a nucleotide with a hydroxyl group at the 2′ position of a β-D-ribo-furanose moiety.

By “nucleic acid decoy molecule”, or “decoy” as used herein is meant a nucleic acid molecule that mimics the natural binding domain for a ligand. The decoy therefore competes with the natural binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., 1990, Cell, 63, 601-608).

By “aptamer” or “nucleic acid aptamer” as used herein is meant a nucleic acid molecule that binds specifically to a target molecule wherein the nucleic acid molecule has sequence that is distinct from sequence recognized by the target molecule in its natural setting. Alternately, an aptamer can be a nucleic acid molecule that binds to a target molecule where the target molecule does not naturally bind to a nucleic acid. The target molecule can be any molecule of interest.

For example, the aptamer can be used to bind to a ligand binding domain of a protein, thereby preventing interaction of the naturally occurring ligand with the protein. Similarly, the nucleic acid molecules of the instant invention can bind to VEGFR1 or VEGF2 receptors to block activity of the receptor. This is a non-limiting example and those in the art will recognize that other embodiments can be readily generated using techniques generally known in the art, see for example Gold et al., U.S. Pat. No. 5,475,096 and 5,270,163; Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628.

The term “double stranded RNA” or “dsRNA” as used herein refers to a double stranded RNA molecule capable of RNA interference “RNAi”, including short interfering RNA “siRNA” see for example Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914. In addition, as used herein, the term RNAi is meant to be equivalent to other terms used to describe sequence specific RNA interference, such as post transciptional gene silencing.

The term “short interfering RNA”, “siRNA”, “short interfering nucleic acid”, “siNA”, “short interfering nucleic acid molecule”, “short interfering oligonucleotide molecule”, or “chemically-modified short interfering nucleic acid moleule” as used herein refers to any nucleic acid molecule capable of mediating RNA interference “RNAi” or gene silencing. For example the siRNA can be a double-stranded polynucleotide molecule comprising self-complementary sense and antisense regions, wherein the antisense region comprises complementarity to a target nucleic acid molecule. The siRNA can be a single-stranded hairpin polynucleotide having self-complementary sense and antisense regions, wherein the antisense region comprises complementarity to a target nucleic acid molecule. The siRNA can be a circular single-stranded polynucleotide having two or more loop structures and a stem comprising self-complementary sense and antisense regions, wherein the antisense region comprises complementarity to a target nucleic acid molecule, and wherein the circular polynucleotide can be processed either in vivo or in vitro to generate an active siRNA capable of mediating RNAi. The siRNA can also comprise a single stranded polynucleotide having complementarity to a target nucleic acid molecule, wherein the single stranded polynucleotide can further comprise a terminal phosphate group, such as a 5′-phosphate (see for example Martinez et al., 2002, Cell., 110, 563-574), or 5′,3′-diphosphate.

As used herein, siRNA molecules need not be limited to those molecules containing only RNA, but further encompasses chemically-modified nucleotides and non-nucleotides. In certain embodiments, the short interfering nucleic acid molecules of the invention lack 2′-hydroxy (2′-OH) containing nucleotides. In certain embodiments the invention features short interfering nucleic acids that do not require the presence of nucleotides having a 2′-hydroxy group for mediating RNAi and as such, short interfering nucleic acid molecules of the invention optionally do not contain any ribonucleotides (e.g., nucleotides having a 2′-OH group). Optionally, siRNA molecules can contain about 5, 10, 20, 30, 40, or 50% ribonucleotides. The modified short interfering nucleic acid molecules of the invention can also be referred to as short interfering modified oligonucleotides “siMON.” As used herein, the term siRNA is meant to be equivalent to other terms used to describe nucleic acid molecules that are capable of mediating sequence specific RNAi, for double-stranded RNA (dsRNA), micro-RNA, short hairpin RNA (shRNA), short interfering oligonucleotide, short interfering nucleic acid, short interfering modified oligonucleotide, chemically-modified siRNA, post-transcriptional gene silencing RNA (ptgsRNA), and others.

By “nucleic acid sensor molecule” or “allozyme” as used herein is meant a nucleic acid molecule comprising an enzymatic domain and a sensor domain, where the ability of the enzymatic nucleic acid domain to catalyze a chemical reaction is dependent on the interaction with a target signaling molecule, such as a nucleic acid, polynucleotide, oligonucleotide, peptide, polypeptide, or protein, for example VEGF, VEGFR1 and/or VEGFR2. The introduction of chemical modifications, additional functional groups, and/or linkers, to the nucleic acid sensor molecule can provide enhanced catalytic activity of the nucleic acid sensor molecule, increased binding affinity of the sensor domain to a target nucleic acid, and/or improved nuclease/chemical stability of the nucleic acid sensor molecule, and are hence within the scope of the present invention (see for example Usman et al., U.S. patent application Ser. No. 09/877,526, George et al., U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No. WO 00/24931, Breaker et al., International PCT Publication Nos. WO 00/26226 and 98/27104, and Sullenger et al., U.S. patent application Ser. No. 09/205,520).

By “sensor component” or “sensor domain” of the nucleic acid sensor molecule as used herein is meant, a nucleic acid sequence (e.g., RNA or DNA or analogs thereof) which interacts with a target signaling molecule, for example, a nucleic acid sequence in one or more regions of a target nucleic acid molecule or more than one target nucleic acid molecule, and which interaction causes the enzymatic nucleic acid component of the nucleic acid sensor molecule to either catalyze a reaction or stop catalyzing a reaction. In the presence of target signaling molecule of the invention, such as VEGF, VEGFR1 and/or VEGFR2, the ability of the sensor component, for example, to modulate the catalytic activity of the nucleic acid sensor molecule, is inhibited or diminished. The sensor component can comprise recognition properties relating to chemical or physical signals capable of modulating the nucleic acid sensor molecule via chemical or physical changes to the structure of the nucleic acid sensor molecule. The sensor component can be derived from a naturally occurring nucleic acid binding sequence, for example, RNAs that bind to other nucleic acid sequences in vivo. Alternately, the sensor component can be derived from a nucleic acid molecule (aptamer) which is evolved to bind to a nucleic acid sequence within a target nucleic acid molecule (see for example Gold et al., U.S. Pat. Nos. 5,475,096 and 5,270,163). The sensor component can be covalently linked to the nucleic acid sensor molecule, or can be non-covalently associated. A person skilled in the art will recognize that all that is required is that the sensor component is able to selectively inhibit the activity of the nucleic acid sensor molecule to catalyze a reaction.

By “target molecule” or “target signaling molecule” is meant a molecule capable of interacting with a nucleic acid sensor molecule, specifically a sensor domain of a nucleic acid sensor molecule, in a manner that causes the nucleic acid sensor molecule to be active or inactive. The interaction of the signaling agent with a nucleic acid sensor molecule can result in modification of the enzymatic nucleic acid component of the nucleic acid sensor molecule via chemical, physical, topological, or conformational changes to the structure of the molecule, such that the activity of the enzymatic nucleic acid component of the nucleic acid sensor molecule is modulated, for example is activated or deactivated. Signaling agents can comprise target signaling molecules such as macromolecules, ligands, small molecules, metals and ions, nucleic acid molecules including but not limited to RNA and DNA or analogs thereof, proteins, peptides, antibodies, polysaccharides, lipids, sugars, microbial or cellular metabolites, pharmaceuticals, and organic and inorganic molecules in a purified or unpurified form, for example VEGF, VEGFR1 and/or VEGFR2.

The term “triplex forming oligonucleotides” as used herein refers to an oligonucleotide that can bind to a double-stranded DNA in a sequence-specific manner to form a triple-strand helix. Formation of such a triple helix structure has been shown to inhibit transcription of a targeted gene (Duval-Valentin et al., 1992 Proc. Natl. Acad. Sci. USA 89, 504; Fox, 2000, Curr. Med. Chem., 7, 17-37; Praseuth et. al., 2000, Biochim. Biophys. Acta, 1489, 181-206).

The nucleic acid molecules that modulate the expression of VEGF and/or VEGFr, such as VEGFR1 and/or VEGFR2 specific nucleic acids, represent a novel therapeutic approach to treat or control a variety of female reproductive disorders and conditions, including but not limited to endometriosis, endometrial carcinoma, gynecologic bleeding disorders, irregular menstrual cycles, ovulation, premenstrual syndrome (PMS), and/or menopausal dysfunction. The nucleic acid molecules that modulate the expression of VEGF and/or VEGFr, such as VEGFR1 and/or VEGFR2 specific nucleic acids also represent a novel approach to control ovulation or embryonic implantation and therefore provide a novel means of birth control.

In one embodiment of the present invention, a nucleic acid molecule of the instant invention can be between 12 and 100 nucleotides in length. An exemplary enzymatic nucleic acid molecule of the invention is shown as Formula I. For example, in one embodiment, the enzymatic nucleic acid molecules of the invention are between 15 and 50 nucleotides in length, including, for example, between 25 and 40 nucleotides in length, e.g., 34, 36, or 38 nucleotides in length (for example see Jarvis et al., 1996, J. Biol. Chem., 271, 29107-29112). In one embodiment, exemplary DNAzymes of the invention are between 15 and 40 nucleotides in length, including, for example, between 25 and 35 nucleotides in length, e.g., 29, 30, 31, or 32 nucleotides in length (see for example Santoro et al., 1998, Biochemistry, 37, 13330-13342; Chartrand et al., 1995, Nucleic Acids Research, 23, 4092-4096). In one embodiment, exemplary antisense molecules of the invention are between 15 and 75 nucleotides in length, including, for example, between 20 and 35 nucleotides in length, e.g., 25, 26, 27, or 28 nucleotides in length (see for example Woolf et al., 1992, PNAS., 89, 7305-7309; Milner et al., 1997, Nature Biotechnology, 15, 537-541). In one embodiment, exemplary triplex forming oligonucleotide molecules of the invention are between 10 and 40 nucleotides in length, including, for example, between 12 and 25 nucleotides in length, e.g., 18, 19, 20, or 21 nucleotides in length (see for example Maher et al., 1990, Biochemistry, 29, 8820-8826; Strobel and Dervan, 1990, Science, 249, 73-75). Those skilled in the art will recognize that all that is required is that the nucleic acid molecule be of length and conformation sufficient and suitable for the nucleic acid molecule to catalyze a reaction contemplated herein. The length of the nucleic acid molecules of the instant invention are not limiting within the general limits stated.

In one embodiment, a nucleic acid molecule that modulates, for example, down-regulates, VEGF and/or VEGFr, such as VEGFR1 and/or VEGFR2, expression or activity comprises between 8 and 100 bases complementary to a nucleic acid molecule of VEGFR1 and/or VEGFR2. In another embodiment, a nucleic acid molecule that modulates VEGF and/or VEGFr, such as VEGFRI and/or VEGFR2 expression or activity comprises between 14 and 24 bases complementary to a nucleic acid molecule of VEGFR1 and/or VEGFR2.

The invention provides a method for producing a class of nucleic acid-based gene modulating agents which exhibit a high degree of specificity for the nucleic acid of a desired target. For example, a nucleic acid molecule of the invention is preferably targeted to a highly conserved sequence region of target nucleic acids encoding VEGF and/or VEGFr, such as VEGFR1 and/or VEGFR2 (specifically VEGF, VEGFRI and/or VEGFR2 genes) such that specific treatment of a disease or condition can be provided with either one or several nucleic acid molecules of the invention. Such nucleic acid molecules can be delivered exogenously to specific tissue or cellular targets as required. Alternatively, the nucleic acid molecules can be expressed from DNA and/or RNA vectors that are delivered to specific cells.

As used in herein “cell” is used in its usual biological sense, and does not refer to an entire multicellular organism. The cell can, for example, be in vitro, e.g., in cell culture, or present in a multicellular organism, including, e.g., birds, plants and mammals, such as humans, cows, sheep, apes, monkeys, swine, dogs, and cats. The cell can be prokaryotic (e.g., bacterial cell) or eukaryotic (e.g., mammalian or plant cell).

By “VEGFR1 and/or VEGFR2 proteins” is meant, protein receptor or a mutant protein derivative thereof, having vascular endothelial growth factor receptor activity, for example, having the ability to bind vascular endothelial growth factor and/or having tyrosine kinase activity.

By “highly conserved sequence region” is meant, a nucleotide sequence of one or more regions in a target gene does not vary significantly from one generation to the other or from one biological system to the other.

Nucleic acid-based inhibitors of VEGF and/or VEGFr, such as VEGFR1 and/or VEGFR2 expression are useful for the prevention, treatment, amelioration and/or control of female reproductive disorders and conditions, including but not limited to endometriosis, endometrial carcinoma, gynecologic bleeding disorders, irregular menstrual cycles, ovulation, premenstrual syndrome (PMS), menopausal dysfunction, and any other diseases or conditions that are related to or will respond to the levels of VEGF, VEGFR1 and/or VEGFR2 in a cell or tissue, alone or in combination with other therapies. The reduction of VEGF and/or VEGFr, such as VEGFR1 and/or VEGFR2 expression (specifically VEGF, VEGFR1 and/or VEGFR2 gene RNA levels) and thus reduction in the level of the respective protein relieves, to some extent, the symptoms of the disease or condition. Nucleic acid-based inhibitors of VEGF and/or VEGFr, such as VEGFR1 and/or VEGFR2 expression are also useful as birth control agents, for example, by inhibition of ovulation or embryonic uterine implantation.

The nucleic acid molecules of the invention can be added directly, or can be complexed with cationic lipids, packaged within liposomes, or otherwise delivered to target cells or tissues. The nucleic acid complexes can be locally administered to relevant tissues ex vivo, or in vivo through injection or infusion pump, with or without their incorporation in biopolymers. In some embodiments, the nucleic acid molecules comprise sequences, which are complementary to polynucleotides, for example DNA and RNA having VEGF and/or VEGFr encoding sequence, such as VEGFR1 and/or VEGFR2 mRNA sequence.

Triplex molecules of the invention can be provided targeted to DNA target regions, and containing the DNA equivalent of a target sequence or a sequence complementary to the specified target (substrate) sequence. Antisense molecules typically are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both.

By “consists essentially of” is meant that the active nucleic acid molecule of the invention, for example, an enzymatic nucleic acid molecule, contains an enzymatic center or core equivalent to those in the examples, and binding arms able to bind nucleic acid such that cleavage at the target site occurs. Other sequences can be present which do not interfere with such cleavage.

Thus, a core region can, for example, include one or more loop, stem-loop structure, or linker which does not prevent enzymatic activity. Thus, a particular region of a nucleic acid molecule of the invention can be such a loop, stem-loop, nucleotide linker, and/or non-nucleotide linker and can be represented generally as sequence “X”. For example, a core sequence for a hammerhead enzymatic nucleic acid can comprise a conserved sequence, such as 5′-CUGAUGAG-3′ and 5′-CGAA-3′ connected by “X”, where X is 5′-GCCGUUAGGC-3′ (SEQ ID NO 12), or any other Stem II region known in the art, or a nucleotide and/or non-nucleotide linker. Similarly, for other nucleic acid molecules of the instant invention, such as Inozyme, G-cleaver, amberzyme, zinzyme, DNAzyme, antisense, 2-5A antisense, triplex forming nucleic acid, aptamers, decoy nucleic acids, dsRNA or siRNA, other sequences or non-nucleotide linkers can be present that do not interfere with the function of the nucleic acid molecule.

Sequence X can be a linker of >2 nucleotides in length, preferably 3, 4, 5, 6, 7, 8, 9, 10, 15, 20, 26, 30, where the nucleotides can preferably be internally base-paired to form a stem of preferably ≧2 base pairs. Alternatively or in addition, sequence X can be a non-nucleotide linker. In yet another embodiment, the nucleotide linker X can be a nucleic acid aptamer, such as an ATP aptamer, HIV Rev aptamer (RRE), HIV Tat aptamer (TAR) and others (for a review see Gold et al., 1995, Annu. Rev. Biochem., 64, 763; and Szostak & Ellington, 1993, in The RNA World, ed. Gesteland and Atkins, pp. 511, CSH Laboratory Press). A nucleic acid aptamer includes a nucleic acid sequence capable of interacting with a ligand. The ligand can be any natural or a synthetic molecule, including but not limited to a resin, metabolites, nucleosides, nucleotides, drugs, toxins, transition state analogs, peptides, lipids, proteins, amino acids, nucleic acid molecules, hormones, carbohydrates, receptors, cells, viruses, bacteria and others.

In yet another embodiment, the non-nucleotide linker X is as defined herein. The term “non-nucleotide” as used herein include either abasic nucleotide, polyether, polyamine, polyamide, peptide, carbohydrate, lipid, or polyhydrocarbon compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO 89/02439; Usman et al, International Publication No. WO 95/06731; Dudycz et al., International Publication No. WO 95/11910 and Ferentz and Verdine, J. Am. Chem. Soc. 1991, 113:4000, all hereby incorporated by reference herein. A “non-nucleotide” further means any group or compound which can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymine. Thus, in a preferred embodiment, the invention features an enzymatic nucleic acid molecule having one or more non-nucleotide moieties, and having enzymatic activity to cleave a RNA or DNA molecule.

In another aspect of the invention, nucleic acid molecules that interact with target nucleic acid molecules and down-regulate VEGF and/or VEGFr, such as VEGFR1 and/or VEGFR2 (specifically VEGF, VEGFR1 and/or VEGFR2 gene) activity are expressed from transcription units inserted into DNA or RNA vectors. The recombinant vectors are preferably DNA plasmids or viral vectors. Enzymatic nucleic acid molecule or antisense expressing viral vectors can be constructed based on, but not limited to, adeno-associated virus, retrovirus, adenovirus, or alphavirus. Preferably, the recombinant vectors capable of expressing the enzymatic nucleic acid molecules or antisense are delivered as described above, and persist in target cells. Alternatively, viral vectors can be used that provide for transient expression of enzymatic nucleic acid molecules or antisense. Such vectors can be repeatedly administered as necessary. Once expressed, the enzymatic nucleic acid molecules or antisense bind to the target nucleic acid and down-regulate its function or expression. Delivery of enzymatic nucleic acid molecule or antisense expressing vectors can be systemic, such as by intravenous or intramuscular administration, by administration to target cells ex-planted from the patient followed by reintroduction into the patient, or by any other means that would allow for introduction into the desired target cell. Antisense DNA can be expressed via the use of a single stranded DNA intracellular expression vector.

By “vectors” is meant any nucleic acid- and/or viral-based technique used to deliver a desired nucleic acid.

By “subject” is meant an organism, which is a donor or recipient of explanted cells, or the cells themselves. “Subject” also refers to an organism to which the nucleic acid molecules of the invention can be administered. A subject can be a mammal or mammalian cells. For example, a subject can be a human or human cells.

By “enhanced enzymatic activity” is meant to include activity measured in cells and/or in vivo where the activity is a reflection of both the catalytic activity and the stability of the nucleic acid molecules of the invention. In this invention, the product of these properties can be increased in vivo compared to an all RNA enzymatic nucleic acid or all DNA enzyme. In some cases, the activity or stability of the nucleic acid molecule can be decreased (i.e., less than ten-fold), but the overall activity of the nucleic acid molecule is enhanced, in vivo.

The nucleic acid molecules of the instant invention, individually, or in combination or in conjunction with other drugs, can be used to treat diseases or conditions discussed above. For example, to treat a disease or condition associated with the levels of VEGFR1 and/or VEGFR2, the patient can be treated, or other appropriate cells can be treated, as is evident to those skilled in the art, individually or in combination with one or more drugs under conditions suitable for the treatment.

In a further embodiment, the described molecules of the invention can be used in combination with other known treatments to treat conditions or diseases discussed above. For example, the described molecules can be used in combination with one or more known therapeutic agents to treat female reproductive disorders and conditions, including but not limited to endometriosis, birth control, endometrial tumors, gynecologic bleeding disorders, irregular menstrual cycles, ovulation, premenstrual syndrome (PMS), menopausal dysfunction, endometrial carcinoma, and/or other diseases or conditions which respond to the modulation of VEGF and/or VEGFr, such as VEGFR1 and/or VEGFR2 expression.

Other features and advantages of the invention will be apparent from the following description of the preferred embodiments thereof, and from the claims.

### BRIEF DESCRIPTION OF THE DRAWINGS

FIG. 1 shows examples of chemically stabilized ribozyme motifs. HH Rz, represents hammerhead ribozyme motif (Usman et al., 1996, Curr. Op. Struct. Bio., 1, 527); NCH Rz represents the NCH ribozyme motif (Ludwig et al., International PCT Publication No. WO 98/58058 and U.S. patent application Ser. No. 08/878,640); G-Cleaver, represents G-cleaver ribozyme motif (Kore et al., 1998, Nucleic Acids Research 26, 4116-4120, Eckstein et al., U.S. Pat. No. 6,127,173). N or n, represent independently a nucleotide which can be same or different and have complementarity to each other; rI, represents ribo-Inosine nucleotide; arrow indicates the site of cleavage within the target. Position 4 of the HH Rz and the NCH Rz is shown as having 2′-C-allyl modification, but those skilled in the art will recognize that this position can be modified with other modifications well known in the art, so long as such modifications do not significantly inhibit the activity of the ribozyme.

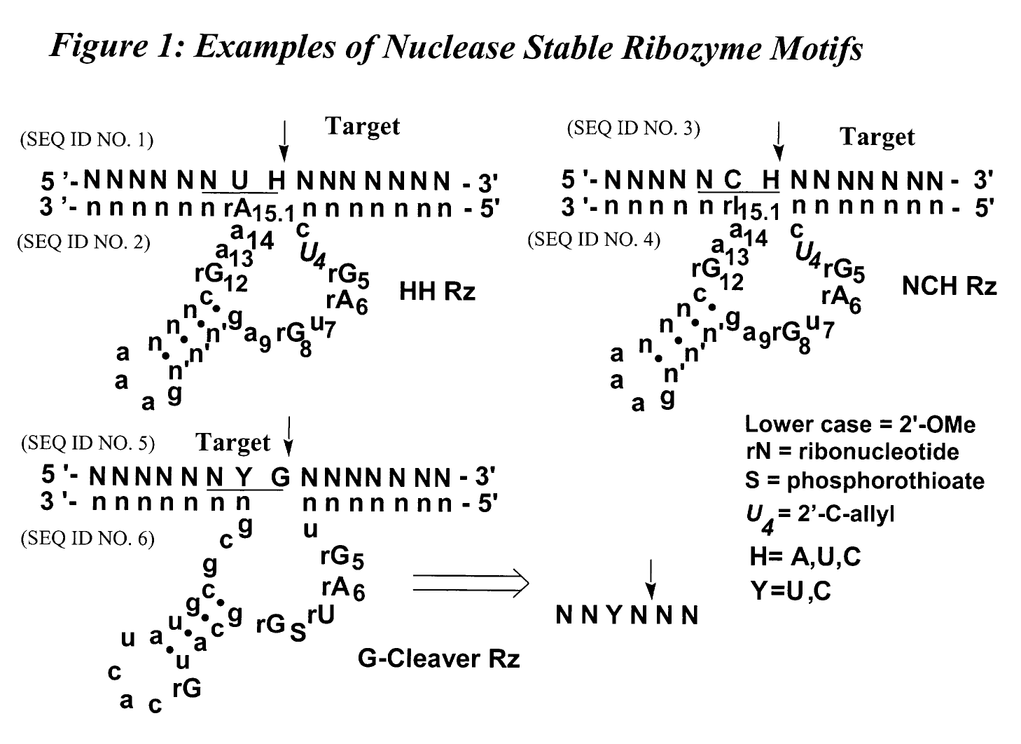

FIG. 2 shows an example of the Amberzyme ribozyme motif that is chemically stabilized (see for example Beigelman et al., International PCT publication No. WO 99/55857 and U.S. patent application Ser. No. 09/476,387.).

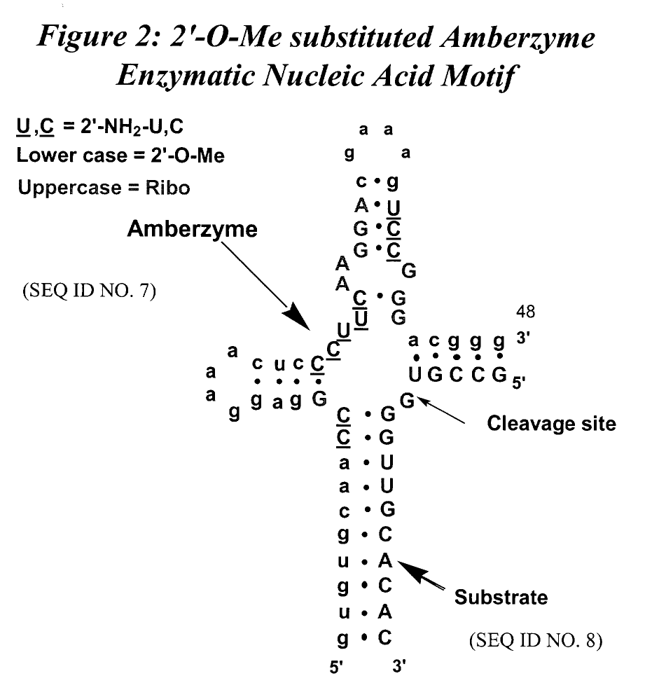

FIG. 3 shows an example of a Zinzyme A ribozyme motif that is chemically stabilized (see for example Beigelman et al., International PCT publication No. WO 99/55857 and U.S. patent application Ser. No. 09/918,728).

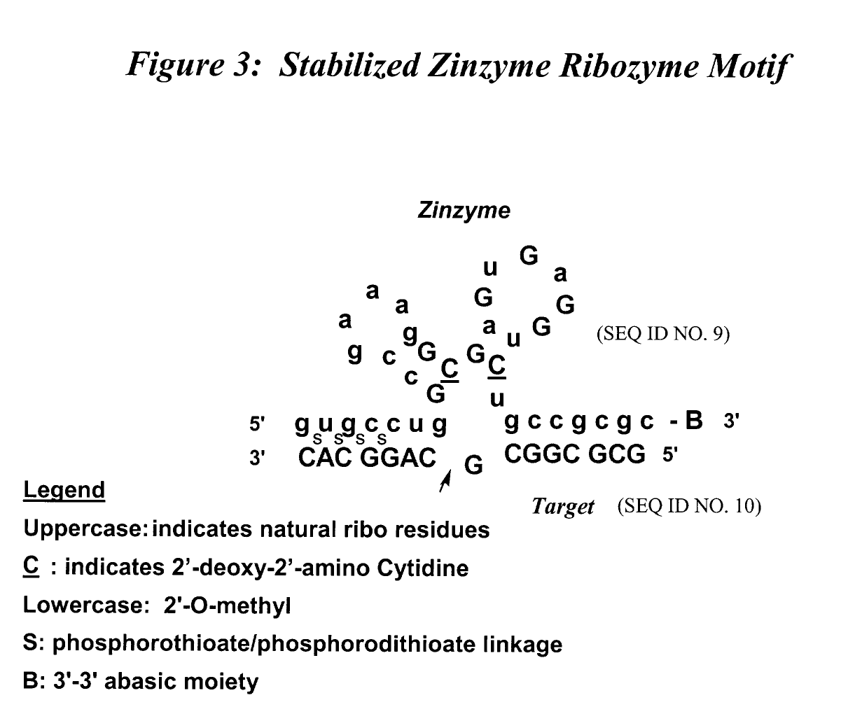

FIG. 4 shows an example of a DNAzyme motif described by Santoro et al., 1997, PNAS, 94, 4262 and Joyce et al., U.S. Pat. No. 5,807,718.

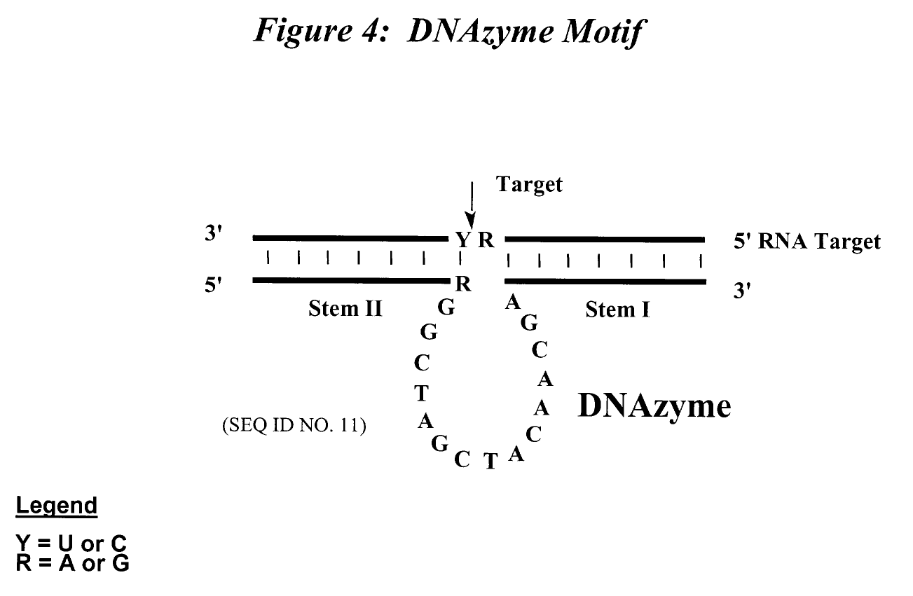

FIG. 5 shows the plasma concentration profile of ANGIOZYME™ after a single SC (sub-cutaneous) dose of 10, 30, 100, or 300 mg/m².

### DESCRIPTION OF THE PREFERRED EMBODIMENTS

Nucleic Acid Molecules and Mechanism of Action

Enzymatic Nucleic Acid: Several varieties of naturally-occurring enzymatic nucleic acids are presently known. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al., 1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other nucleic acid molecules) under physiological conditions.

The enzymatic nature of an enzymatic nucleic acid molecule has significant advantages, one advantage being that the concentration of enzymatic nucleic acid molecule necessary to affect a therapeutic treatment is lower. This advantage reflects the ability of the enzymatic nucleic acid molecule to act enzymatically. Thus, a single enzymatic nucleic acid molecule is able to cleave many molecules of target nucleic acid. In addition, the enzymatic nucleic acid molecule is a highly specific inhibitor, with the specificity of inhibition depending not only on the base-pairing mechanism of binding to the target nucleic acid, but also on the mechanism of target nucleic acid cleavage. Single mismatches, or base-substitutions, near the site of cleavage can be chosen to completely eliminate catalytic activity of a enzymatic nucleic acid molecule.

Nucleic acid molecules having an endonuclease enzymatic activity are able to repeatedly cleave other separate nucleic acid molecules in a nucleotide base sequence-specific manner. With the proper design, such enzymatic nucleic acid molecules can be targeted to RNA transcripts, and achieve efficient cleavage in vitro (Zaug et al., 324, Nature 429 1986; Uhlenbeck, 1987 Nature 328, 596; Kim et al., 84 Proc. Natl. Acad. Sci. USA 8788, 1987; Dreyfus, 1988, Einstein Quart. J Bio. Med., 6, 92; Haseloff and Gerlach, 334 Nature 585, 1988; Cech, 260 JAMA 3030, 1988; and Jefferies et al, 17 Nucleic Acids Research 1371, 1989; Santoro et al., 1997 supra).

Because of their sequence specificity, trans-cleaving enzymatic nucleic acid molecules can be used as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific nucleic acid targets within the background of cellular nucleic acid. Such a cleavage event renders the nucleic acid non-functional and abrogates protein expression from that nucleic acid. In this manner, synthesis of a protein associated with a disease state can be selectively inhibited (Warashina et al., 1999, Chemistry and Biology, 6, 237-250).

Enzymatic nucleic acid molecules of the invention that are allosterically regulated (“allozymes”) can be used to down-regulate VEGF and/or VEGFr, such as VEGFR1 and/or VEGFR2 expression. These allosteric enzymatic nucleic acids or allozymes (see for example Usman et al., U.S. patent application Ser. No. 09/877,526, George et al., U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No. WO 00/24931, Breaker et al., International PCT Publication Nos. WO 00/26226 and 98/27104, and Sullenger et al., U.S. patent application Ser. No. 09/205,520) are designed to respond to a signaling agent, for example, mutant VEGFR1 and/or VEGFR2 protein, wild-type VEGFR1 and/or VEGFR2 protein, mutant VEGFR1 and/or VEGFR2 RNA, wild-type VEGFR1 and/or VEGFR2 RNA, other proteins and/or RNAs involved in VEGF signal transduction, compounds, metals, polymers, molecules and/or drugs that are targeted to VEGFR1 and/or VEGFR2 expressing cells etc., which in turn modulates the activity of the enzymatic nucleic acid molecule. In response to interaction with a predetermined signaling agent, the allosteric enzymatic nucleic acid molecule's activity is activated or inhibited such that the expression of a particular target is selectively down-regulated. The target can comprise wild-type VEGFR1 and/or VEGFR2, mutant VEGFR1 and/or VEGFR2, and/or a predetermined component of the VEGF signal transduction pathway. In a specific example, allosteric enzymatic nucleic acid molecules that are activated by interaction with a RNA encoding VEGF protein are used as therapeutic agents in vivo. The presence of RNA encoding the VEGF protein activates the allosteric enzymatic nucleic acid molecule that subsequently cleaves the RNA encoding a VEGFR1 and/or VEGFR2 protein resulting in the inhibition of VEGFR1 and/or VEGFR2 protein expression.

In another non-limiting example, an allozyme can be activated by a VEGF and/or VEGFr, such as VEGFR1 and/or VEGFR2 protein, peptide, or mutant polypeptide that causes the allozyme to inhibit the expression of VEGF and/or VEGFr, such as VEGFR1 and/or VEGFR2 genes, by, for example, cleaving RNA encoded by VEGF, VEGFR1 and/or VEGFR2 gene. In this non-limiting example, the allozyme acts as a decoy to inhibit the function of VEGF, VEGFR1 and/or VEGFR2 and also inhibit the expression of VEGF, VEGFR1 and/or VEGFR2 once activated by the VEGF, VEGFR1 and/or VEGFR2 protein.

Antisense: Antisense molecules can be modified or unmodified RNA, DNA, or mixed polymer oligonucleotides and primarily function by specifically binding to matching sequences resulting in inhibition of peptide synthesis (Wu-Pong, Nov 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules can also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA can result in nuclease degradation of the heteroduplex (Wu-Pong, supra; Crooke, supra). To date, the only backbone modified DNA chemistry which act as substrates for RNase H are phosphorothioates, phosphorodithioates, and borontrifluoridates. Recently it has been reported that 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., International PCT Publication No. WO 99/54459; Hartmann et al., U.S. S No. 60/101,174 which was filed on Sep. 21, 1998) all of these are incorporated by reference herein in their entirety.

In addition, antisense deoxyoligoribonucleotides can be used to target RNA by means of DNA-RNA interactions, thereby activating RNase H, which digests the target RNA in the duplex. Antisense DNA can be expressed via the use of a single stranded DNA intracellular expression vector or equivalents and variations thereof.

Triplex Forming Oligonucleotides (TFO): Single stranded DNA can be designed to bind to genomic DNA in a sequence specific manner. TFOs are comprised of pyrimidine-rich oligonucleotides which bind DNA helices through Hoogsteen Base-pairing (Wu-Pong, supra).

The resulting triple helix composed of the DNA sense, DNA antisense, and TFO disrupts RNA synthesis by RNA polymerase. The TFO mechanism can result in gene expression or cell death since binding can be irreversible (Mukhopadhyay & Roth, supra).

2-5A Antisense Chimera: The 2-5A system is an interferon mediated mechanism for RNA degradation found in higher vertebrates (Mitra et al., 1996, Proc Nat Acad Sci USA 93, 6780-6785). Two types of enzymes, 2-5A synthetase and RNase L, are required for RNA cleavage.

The 2-5A synthetases require double stranded RNA to form 2′-5′ oligoadenylates (2-5A). 2-5A then acts as an allosteric effector for utilizing RNase L which has the ability to cleave single stranded RNA. The ability to form 2-5A structures with double stranded RNA makes this system particularly useful for inhibition of viral replication.

(2′-5′) oligoadenylate structures can be covalently linked to antisense molecules to form chimeric oligonucleotides capable of RNA cleavage (Torrence, supra). These molecules putatively bind and activate a 2-5A dependent RNase, the oligonucleotide/enzyme complex then binds to a target RNA molecule which can then be cleaved by the RNase enzyme.

RNAi: Double-stranded RNAs can suppress expression of homologous genes through an evolutionarily conserved process named RNA interference (RNAi) or post-transcriptional gene silencing (PTGS). One mechanism underlying silencing is the degradation of target mRNAs by an RNP complex, which contains short interfering RNAs (siRNAs) as guides to substrate selection. Short interfering RNAs are typically 21 to 23 nucleotides in length. A bidentate nuclease called Dicer has been implicated as the protein responsible for siRNA production. For example, a double-stranded RNA (dsRNA) matching a gene sequence is synthesized in vitro and introduced into a cell. The dsRNA feeds into a biological pathway and is broken into short pieces of short interfering (si) RNAs. With the help of cellular enzymes such as Dicer, the siRNA triggers the degradation of the messenger RNA that matches its sequence (see for example Tuschl et al., International PCT Publication No. WO 01/75164; Bass, 2001, Nature, 411, 428-429; Elbashir et al., 2001, Nature, 411, 494-498; and Kreutzer et al., International PCT Publication No. WO 00/44895).

Target Sites

Targets for useful nucleic acid molecules of the invention, such as enzymatic nucleic acid molecules, dsRNA, and antisense nucleic acids can be determined as disclosed in Draper et al., 30 WO 93/23569; Sullivan et al., WO 93/23057; Thompson et al., WO 94/02595; Draper et al., WO 95/04818; McSwiggen et al., U.S. Pat. No. 5,525,468, and hereby incorporated by reference herein in totality. Other examples include the following PCT applications, which concern inactivation of expression of disease-related genes: WO 95/23225, WO 95/13380, WO 94/02595, incorporated by reference herein. Rather than repeat the guidance provided in those documents here, below are provided specific examples of such methods, not limiting to those in the art.

Enzymatic nucleic acid molecules, siRNA and antisense to such targets are designed as described in those applications and synthesized to be tested in vitro and in vivo, as also described. The sequences of human VEGF, VEGFR1 and/or VEGFR2 RNAs are screened for optimal nucleic acid target sites using a computer-folding algorithm. Potential nucleic acid binding/cleavage sites are identified. While human sequences can be screened and nucleic acid molecules thereafter designed, as discussed in Stinchcomb et al., WO 95/23225, mouse targeted enzymatic nucleic acid molecules can be useful to test efficacy of action of the nucleic acid molecule prior to testing in humans.

Nucleic acid molecule binding/cleavage sites are identified, for example enzymatic nucleic acid, antisense, and dsRNA mediated binding sites are chosen. For enzymatic nucleic acid molecules of the invention, the nucleic acid molecules are individually analyzed by computer folding (Jaeger et al., 1989 Proc. NatL Acad. Sci. USA, 86, 7706) to assess whether the sequences fold into the appropriate secondary structure. Those nucleic acid molecules with unfavorable intramolecular interactions such as between the binding arms and the catalytic core can be eliminated from consideration. Varying binding arm lengths can be chosen to optimize activity.

Nucleic acids, such as antisense, RNAi, and/or enzymatic nucleic acid molecule binding/cleavage sites are identified and are designed to anneal to various sites in the nucleic acid target. The binding arms of enzymatic nucleic acid molecules of the invention are complementary to the target site sequences described above. Antisense and RNAi sequences are designed to have partial or complete complementarity to the nucleic acid target. The nucleic acid molecules can be chemically synthesized. The method of synthesis used follows the procedure for normal DNA/RNA synthesis as described below and in Usman et al., 1987 J Am. Chem. Soc., 109, 7845; Scaringe et al., 1990 Nucleic Acids Res., 18, 5433; and Wincott et al., 1995 Nucleic Acids Res. 23, 2677-2684; Caruthers et al., 1992, Methods in Enzymology 211,3-19.

Synthesis of Nucleic acid Molecules

Synthesis of nucleic acids greater than 100 nucleotides in length is difficult using automated methods, and the therapeutic cost of such molecules is prohibitive. In this invention, small nucleic acid motifs (“small refers to nucleic acid motifs less than about 100 nucleotides in length, preferably less than about 80 nucleotides in length, and more preferably less than about 50 nucleotides in length; e.g., antisense oligonucleotides, enzymatic nucleic acids, aptamers, allozymes, decoys, siRNA etc.) are preferably used for exogenous delivery. The simple structure of these molecules increases the ability of the nucleic acid to invade targeted regions of RNA structure. Exemplary molecules of the instant invention are chemically synthesized, and others can similarly be synthesized. Oligonucleotides (eg, DNA) are synthesized using protocols known in the art as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 2.5 min coupling step for 2′-O-methylated nucleotides and a 45 sec coupling step for 2′-deoxy nucleotides. Table II outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 105-fold excess of S-ethyl tetrazole (60 μL of 0.25 M=15 μmol) can be used in each coupling cycle of 2′-O-methyl residues relative to polymer-bound 5′-hydroxyl. A 22-fold excess (40 μL of 0.11 M=4.4 μmol) of deoxy phosphoramidite and a 70-fold excess of S-ethyl tetrazole (40 μL of 0.25 M=10 μmol) can be used in each coupling cycle of deoxy residues relative to polymer-bound 5′-hydroxyl.

Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM 12, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the DNA polynucleotides is performed as follows: the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H20/3:1:1, vortexed and the supernatant is then added to the first supernatant. The combined supernatants, containing the oligoribonucleotide, are dried to a white powder.

The method of synthesis used for RNA including certain nucleic acid molecules of the invention follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by calorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM 12, 49 mM pyridine, 9% water in THF (PERSEPTIVETM). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide 0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H20/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA•3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA-3HF (0.1 mL) is added and the vial is heated at 65° C. for 15 min. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

Inactive hammerhead ribozymes or binding attenuated control (BAC) oligonucleotides) are synthesized by substituting a U for G₅ and a U for A₁₄ (numbering from Hertel, K. J., et al., 1992, Nucleic Acids Res., 20, 3252). Similarly, one or more nucleotide substitutions can be introduced in other enzymatic nucleic acid molecules to inactivate the molecule and such molecules can serve as a negative control.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the example described above including but not limited to 96 well format, all that is important is the ratio of chemicals used in the reaction.

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

Preferably, the nucleic acid molecules of the present invention are modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Ribozymes are purified by gel electrophoresis using general methods or are purified by high pressure liquid chromatography (HPLC; See Wincott et al., Supra, the totality of which is hereby incorporated herein by reference) and are re-suspended in water.

Optimizing Activity of the Nucleic Acid Molecule of the Invention.

Chemically synthesizing nucleic acid molecules with modifications (base, sugar and/or phosphate) that prevent their degradation by serum ribonucleases can increase their potency potency (see e.g., Eckstein et al., International Publication No. WO 92/07065; Perrault et al, 1990 Nature 344, 565; Pieken et al., 1991, Science 253, 314; Usman and Cedergren, 1992, Trends in Biochem. Sci. 17, 334; Usman et al., International Publication No. WO 93/15187; and

Rossi et al., International Publication No. WO 91/03162; Sproat, U.S. Pat. No. 5,334,711; Gold et al., U.S. Pat. No. 6,300,074; and Burgin et al., supra; all of which are incorporated by reference herein).

Modifications which enhance their efficacy in cells, and removal of bases from nucleic acid molecules to shorten oligonucleotide synthesis times and reduce chemical requirements are desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. S No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). Such publications describe general methods and strategies to determine the location of incorporation of sugar, base and/or phosphate modifications and the like into ribozymes without inhibiting catalysis, and are incorporated by reference herein. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, an over-abundance of these modifications can cause toxicity. Therefore, the amount of these internucleotide linkages should be evaluated and appropriately minimized when designing the nucleic acid molecules. The reduction in the concentration of these linkages should lower toxicity resulting in increased efficacy and higher specificity of these molecules.

Nucleic acid molecules having chemical modifications that maintain or enhance activity are provided. Such nucleic acid molecules are also generally more resistant to nucleases than unmodified nucleic acid. Thus, in a cell and/or in vivo the activity may not be significantly lowered. Therapeutic nucleic acid molecules delivered exogenously are optimally stable within cells until translation of the target RNA has been inhibited long enough to reduce the levels of the undesirable protein. This period of time varies between hours to days depending upon the disease state. Clearly, nucleic acid molecules must be resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211,3-19 (incorporated by reference herein) have expanded the ability to modify nucleic acid molecules by introducing nucleotide modifications to enhance their nuclease stability as described above.

In one embodiment, nucleic acid molecules of the invention include one or more G-clamp nucleotides. A G-clamp nucleotide is a modified cytosine analog wherein the modifications confer the ability to hydrogen bond both Watson-Crick and Hoogsteen faces of a complementary guanine within a duplex, see for example Lin and Matteucci, 1998, J. Am. Chem. Soc., 120, 8531-8532. A single G-clamp analog substitution within an oligonucleotide can result in substantially enhanced helical thermal stability and mismatch discrimination when hybridized to complementary oligonucleotides. The inclusion of such nucleotides in nucleic acid molecules of the invention results in both enhanced affinity and specificity to nucleic acid targets. In another embodiment, nucleic acid molecules of the invention include one or more LNA “locked nucleic acid” nucleotides such as a 2′, 4′-C mythylene bicyclo nucleotide (see for example Wengel et al., International PCT Publication No. WO 00/66604 and WO 99/14226).

In another embodiment, the invention features conjugates and/or complexes of nucleic acid molecules targeting VEGF receptors such as VEGFR1 and/or VEGFR2. Such conjugates and/or complexes can be used to facilitate delivery of molecules into a biological system, such as cells. The conjugates and complexes provided by the instant invention can impart therapeutic activity by transferring therapeutic compounds across cellular membranes, altering the pharmacokinetics, and/or modulating the localization of nucleic acid molecules of the invention.

The present invention encompasses the design and synthesis of novel conjugates and complexes for the delivery of molecules, including but not limited to small molecules, lipids, phospholipids, nucleosides, nucleotides, nucleic acids, antibodies, toxins, negatively charged polymers and other polymers, for example proteins, peptides, hormones, carbohydrates, polyethylene glycols, or polyamines, across cellular membranes. In general, the transporters described are designed to be used either individually or as part of a multi-component system, with or without degradable linkers. These compounds are expected to improve delivery and/or localization of nucleic acid molecules of the invention into a number of cell types originating from different tissues, in the presence or absence of serum (see Sullenger and Cech, U.S. Pat. No. 5,854,038). Conjugates of the molecules described herein can be attached to biologically active molecules via linkers that are biodegradable, such as biodegradable nucleic acid linker molecules.

The term “biodegradable nucleic acid linker molecule” as used herein, refers to a nucleic acid molecule that is designed as a biodegradable linker to connect one molecule to another molecule, for example, a biologically active molecule. The stability of the biodegradable nucleic acid linker molecule can be modulated by using various combinations of ribonucleotides, deoxyribonucleotides, and chemically modified nucleotides, for example, 2′-O-methyl, 2′-fluoro, 2′-amino, 2′-O-amino, 2′-C-allyl, 2′-O-allyl, and other 2′-modified or base modified nucleotides. The biodegradable nucleic acid linker molecule can be a dimer, trimer, tetramer or longer nucleic acid molecule, for example, an oligonucleotide of about 2, 3, 4, 5, 6, 7, 8, 9, 10, 11, 12, 13, 14, 15, 16, 17, 18, 19, or 20 nucleotides in length, or can comprise a single nucleotide with a phosphorus based linkage, for example, a phosphoramidate or phosphodiester linkage. The biodegradable nucleic acid linker molecule can also comprise nucleic acid backbone, nucleic acid sugar, or nucleic acid base modifications.

The term “biodegradable” as used herein, refers to degradation in a biological system, for example enzymatic degradation or chemical degradation.

The term “biologically active molecule” as used herein, refers to compounds or molecules that are capable of eliciting or modifying a biological response in a system. Non-limiting examples of biologically active molecules contemplated by the instant invention include therapeutically active molecules such as antibodies, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, siRNA, dsRNA, allozymes, aptamers, decoys and analogs thereof. Biologically active molecules of the invention also include molecules capable of modulating the pharmacokinetics and/or pharmacodynamics of other biologically active molecules, for example, lipids and polymers such as polyamines, polyamides, polyethylene glycol and other polyethers.

The term “phospholipid” as used herein, refers to a hydrophobic molecule comprising at least one phosphorus group. For example, a phospholipid can comprise a phosphorus containing group and saturated or unsaturated alkyl group, optionally substituted with OH, COOH, oxo, amine, or substituted or unsubstituted aryl groups.

Therapeutic nucleic acid molecules, such as the molecules described herein, delivered exogenously are optimally stable within cells until translation of the target RNA has been inhibited long enough to reduce the levels of the undesirable protein. This period of time varies between hours to days depending upon the disease state. These nucleic acid molecules should be resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

In another embodiment, nucleic acid catalysts having chemical modifications that maintain or enhance enzymatic activity are provided. Such nucleic acids are also generally more resistant to nucleases than unmodified nucleic acid. Thus, in a cell and/or in vivo the activity of the nucleic acid may not be significantly lowered. As exemplified herein such enzymatic nucleic acids are useful in a cell and/or in vivo even if activity over all is reduced 10 fold (Burgin et al., 1996, Biochemistry, 35, 14090). Such enzymatic nucleic acids herein are said to “maintain” the enzymatic activity of an all RNA ribozyme or all DNA DNAzyme.

In another aspect the nucleic acid molecules comprise a 5′ and/or a 3′-cap structure.

By “cap structure” is meant chemical modifications, which have been incorporated at either terminus of the oligonucleotide (see for example Wincott et al., WO 97/26270, incorporated by reference herein). These terminal modifications protect the nucleic acid molecule from exonuclease degradation, and can help in delivery and/or localization within a cell. The cap can be present at the 5′-terminus (5′-cap) or at the 3′-terminus (3′-cap) or can be present on both terminus. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et aL, International PCT publication No. WO 97/26270, incorporated by reference herein).

In another embodiment the 3′-cap includes, for example 4′,5′-methylene nucleotide; 1-(bela-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threopentofuranosy nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

An “alkyl” group refers to a saturated aliphatic hydrocarbon, including straight-chain, branched-chain, and cyclic alkyl groups. Preferably, the alkyl group has 1 to 12 carbons. More preferably it is a lower alkyl of from 1 to 7 carbons, more preferably 1 to 4 carbons. The alkyl group can be substituted or unsubstituted. When substituted the substituted group(s) is preferably, hydroxyl, cyano, alkoxy, ═O, ═S, NO₂ or N(CH₃)₂, amino, or SH. The term also includes alkenyl groups which are unsaturated hydrocarbon groups containing at least one carbon-carbon double bond, including straight-chain, branched-chain, and cyclic groups. Preferably, the alkenyl group has 1 to 12 carbons. More preferably it is a lower alkenyl of from 1 to 7 carbons, more preferably 1 to 4 carbons. The alkenyl group can be substituted or unsubstituted. When substituted the substituted group(s) is preferably, hydroxyl, cyano, alkoxy, ═O, ═S, NO₂, halogen, N(CH₃)₂, amino, or SH. The term “alkyl” also includes alkynyl groups which have an unsaturated hydrocarbon group containing at least one carbon-carbon triple bond, including straight-chain, branched-chain, and cyclic groups. Preferably, the alkynyl group has 1 to 12 carbons. More preferably it is a lower alkynyl of from 1 to 7 carbons, more preferably 1 to 4 carbons. The alkynyl group can be substituted or unsubstituted. When substituted the substituted group(s) is preferably, hydroxyl, cyano, alkoxy, ═O, ═S, NO₂ or N(CH₃)₂, amino or SH.

Such alkyl groups can also include aryl, alkylaryl, carbocyclic aryl, heterocyclic aryl, amide and ester groups. An “aryl” group refers to an aromatic group which has at least one ring having a conjugated p electron system and includes carbocyclic aryl, heterocyclic aryl and biaryl groups, all of which can be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH —R, where R is either alkyl, aryl, alkylaryl or hydrogen. An “ester” refers to an —C(O)—OR′, where R is either alkyl, aryl, alkylaryl or hydrogen.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et aL., International PCT Publication No. WO 93/15187; Uhlman & Peyman, supra all are hereby incorporated by reference herein). There are several examples of modified nucleic acid bases known in the art as summarized by Limbach et aL, 1994, Nucleic Acids Res. 22, 2183. Some of the non-limiting examples of chemically modified and other natural nucleic acid bases that can be introduced into nucleic acids include, for example, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

By “nucleoside” is meant a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, modified nucleosides, non-natural nucleosides, non-standard nucleosides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleoside bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

In one embodiment, the invention features modified enzymatic nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

By “abasic” is meant sugar moieties lacking a base or having other chemical groups in place of a base at the 1′ position, for example a 3′,3′-linked or 5′,5′-linked deoxyabasic ribose derivative (for more details see Wincott et al., International PCT publication No. WO 97/26270).

By “unmodified nucleoside” is meant one of the bases adenine, cytosine, guanine, thymine, uracil joined to the 1′ carbon of β-D-ribo-furanose.

By “modified nucleoside” is meant any nucleotide base which contains a modification in the chemical structure of an unmodified nucleotide base, sugar and/or phosphate.

In connection with 2′-modified nucleotides as described for the present invention, by “amino” is meant 2′-NH₂ or 2′-O—NH₂, which can be modified or unmodified. Such modified groups are described, for example, in Eckstein et al., U.S. Pat. No. 5,672,695 and Matulic-Adamic et al., WO 98/28317, respectively, which are both incorporated by reference in their entireties.

Various modifications to nucleic acid structure can be made to enhance the utility of these molecules. For example, such modifications can enhance shelf-life, half-life in vitro, stability, and ease of introduction of such oligonucleotides to the target site, including e.g., enhancing penetration of cellular membranes and conferring the ability to recognize and bind to targeted cells.

Use of the nucleic acid-based molecules of the invention can lead to better treatment of the disease progression by affording the possibility of combination therapies (e.g., multiple enzymatic nucleic acid molecules targeted to different genes, enzymatic nucleic acid molecules coupled with known small molecule inhibitors, or intermittent treatment with combinations of enzymatic nucleic acid molecules (including different enzymatic nucleic acid molecule motifs) and/or other chemical or biological molecules). The treatment of patients with nucleic acid molecules can also include combinations of different types of nucleic acid molecules. Therapies can be devised which include a mixture of enzymatic nucleic acid molecules (including different enzymatic nucleic acid molecule motifs), allozymes, antisense, dsRNA, aptamers, and/or 2-5A chimera molecules to one or more targets to alleviate symptoms of a disease.

Administration of Nucleic Acid Molecules

Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995 which are both incorporated herein by reference. Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to oral (tablet or pill form) and/or intrathecal delivery (Gold, 1997, Neuroscience, 76, 1153-1158). Other approaches include the use of various transport and carrier systems, for example though the use of conjugates and biodegradable polymers. For a comprehensive review on drug delivery strategies including CNS delivery, see Ho et al., 1999, Curr. Opin. Mol. Ther., 1, 336-343 and Jain, Drug Delivery Systems: Technologies and Commercial Opportunities, Decision Resources, 1998 and Groothuis et al., 1997, J. NeuroVirol., 3, 387-400. More detailed descriptions of nucleic acid delivery and administration are provided in Sullivan et al., supra, Draper et al., PCT WO93/23569, Beigelman et al., PCT WO99/05094, and Klimuk et al., PCT WO99/04819 all of which have been incorporated by reference herein.

The molecules of the instant invention can be used as pharmaceutical agents.

Pharmaceutical agents prevent, inhibit the occurrence, or treat (alleviate a symptom to some extent, preferably all of the symptoms) of a disease state in a patient. The polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, preferably a human. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cells implicated in endometriosis, birth control, endometrial tumors, gynecologic bleeding disorders, irregular menstrual cycles, ovulation, premenstrual syndrome (PMS), menopausal dysfunction, and endometrial carcinoma.

By pharmaceutically acceptable formulation is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, DF et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies, including CNS delivery of the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. All these references are hereby incorporated herein by reference.

The invention also features the use of the composition comprising surface-modified liposomes containing poly (ethylene glycol) lipids (PEG-modified, or long-circulating liposomes or stealth liposomes). Nucleic acid molecules of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al, International PCT Publication No. WO 96/10390; Holland et al., International PCT Publication No. WO 96/10392; all of which are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono-or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per patient per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a patient in combination with other therapeutic compounds to increase the overall therapeutic effect. The use of multiple compounds to treat an indication can increase the beneficial effects while reducing the presence of side effects.

Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45; all of these references are hereby incorporated in their totalities by reference herein). Those skilled in the art realize that any nucleic acid can be expressed in eukaryotic cells from the appropriate DNA/RNA vector. The activity of such nucleic acids can be augmented by their release from the primary transcript by a enzymatic nucleic acid (Draper et al, PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856; all of these references are hereby incorporated in their totalities by reference herein). Gene therapy approaches specific to the CNS are described by Blesch et al., 2000, Drug News Perspect., 13, 269-280; Peterson et al., 2000, Cent. Nerv. Syst. Dis., 485-508; Peel and Klein, 2000, J. Neurosci. Methods, 98, 95-104; Hagihara et al., 2000, Gene Ther., 7, 759-763; and Herrlinger et al., 2000, Methods Mol. Med., 35, 287-312. AAV-mediated delivery of nucleic acid to cells of the nervous system is further described by Kaplitt et al., U.S. Pat. No. 6,180,613.

In another aspect of the invention, RNA molecules of the present invention are preferably expressed from transcription units (see for example Couture et al., 1996, TIG., 12, 510) inserted into DNA or RNA vectors. The recombinant vectors are preferably DNA plasmids or viral vectors. Ribozyme expressing viral vectors can be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors can be used that provide for transient expression of nucleic acid molecules. Such vectors can be repeatedly administered as necessary. Once expressed, the nucleic acid molecule binds to the target mRNA. Delivery of nucleic acid molecule expressing vectors can be systemic, such as by intravenous or intramuscular administration, by administration to target cells ex-planted from the patient followed by reintroduction into the patient, or by any other means that would allow for introduction into the desired target cell (for a review see Couture et al., 1996, TIG., 12, 510).

In one aspect the invention features an expression vector comprising a nucleic acid sequence encoding at least one of the nucleic acid molecules of the instant invention. The nucleic acid sequence encoding the nucleic acid molecule of the instant invention is operably linked in a manner which allows expression of that nucleic acid molecule. In another aspect the invention features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant invention; and wherein said sequence is operably linked to said initiation region and said termination region, in a manner which allows expression and/or delivery of said nucleic acid molecule. The vector can optionally include an open reading frame (ORF) for a protein operably linked on the 5′ side or the 3′-side of the sequence encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743-7; Gao and Huang 1993, Nucleic Acids Res., 21, 2867-72; Lieber et al., 1993, Methods Enzymol., 217, 47-66; Zhou et al., 1990, Mol. Cell. Biol., 10, 4529-37). All of these references are incorporated by reference herein. Several investigators have demonstrated that nucleic acid molecules, such as ribozymes expressed from such promoters can function in mammalian cells (e.g. Kashani-Sabet et al., 1992, Antisense Res. Dev., 2, 3-15; Ojwang et al., 1992, Proc. Natl. Acad. Sci. USA, 89, 10802-6; Chen et al., 1992, Nucleic Acids Res., 20, 4581-9; Yu et al., 1993, Proc. Natl. Acad. Sci. USA, 90, 6340-4; L'Huillier et aL, 1992, EMBO J, 11, 4411-8; Lisziewicz et al., 1993, Proc. Natl. Acad. Sci. U.S.A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736; all of these publications are incorporated by reference herein. The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In another aspect the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule.

In another embodiment the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule. In yet another embodiment the expression vector comprises: a) a transcription initiation region; b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule.

In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

### EXAMPLES

The following are non-limiting examples showing the selection, isolation, synthesis and activity of nucleic acids of the instant invention.

The following examples demonstrate the selection and design of antisense, aptamer, dsRNA, allozyme, hammerhead, DNAzyme, NCH, Amberzyme, Zinzyme, or G-Cleaver ribozyme molecules and binding/cleavage sites within VEGF, VEGFR1 and/or VEGFR2 RNA.

### EXAMPLE 1

### Identification of Potential Target Sites in Human VEGFR1 and/or VEGFR2 RNA

The sequence of human VEGFR1 and/or VEGFR2 genes are screened for accessible sites using a computer-folding algorithm. Regions of the RNA that do not form secondary folding structures and contain potential enzymatic nucleic acid molecule and/or antisense binding/cleavage sites are identified. An exemplary sequence of an enzymatic nucleic acid molecule of the invention is shown in Formula I. Other nucleic acid molecules and targets contemplated by the invention are described in Pavco et al., U.S. patent application Ser. No. 09/870,161, incorporated by reference herein in its entirety. Similarly, other nucleic acid molecules of the invention, including antisense, aptamers, dsRNA, siRNA, and/or 2,5-A chimeras, can be designed to modulate the expression of the nucleic acid targets described in Pavco et al., U.S. patent application Ser. No. 09/870,161.

### EXAMPLE 2

### Selection of Enzymatic Nucleic Acid Cleavage Sites in Human VEGFR1 and/or VEGFR2 RNA

Enzymatic nucleic acid molecule target sites are chosen by analyzing sequences of human VEGFR1 receptor (for example Genbank Accession No. NM_{—}002019), and VEGFR2 receptor (for example Genbank Accession No. NM_{—}002253) genes and prioritizing the sites on the basis of folding. Enzymatic nucleic acid molecules are designed that can bind each target and are individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the enzymatic nucleic acid molecule sequences fold into the appropriate secondary structure. Those enzymatic nucleic acid molecules with unfavorable intramolecular interactions between the binding arms and the catalytic core can be eliminated from consideration. As noted below, varying binding arm lengths can be chosen to optimize activity. Generally, at least 4 bases on each arm are able to bind to, or otherwise interact with, the target RNA.

### EXAMPLE 3

### Chemical Synthesis and Purification of Ribozymes and Antisense for Efficient Cleavage and/or Blocking of VEGFR1 and/or VEGFR2 RNA

Enzymatic nucleic acid molecules and antisense constructs are designed to anneal to various sites in the RNA message. The binding arms of the enzymatic nucleic acid molecules are complementary to the target site sequences described above, while the antisense constructs are fully complementary to the target site sequences described above. RNAi molecules (dsRNA) likewise have one strand of RNA or a portion of RNA complementarity to the target site sequence or a portion of the target site sequence. For example, complementary within the double-strand RNAi structure is formed from two separate individual RNA strands or from self-complementary areas of a topologically closed, individual RNA strand which can be optionally circular. The nucleic acid molecules are chemically synthesized. The method of synthesis used followed the procedure for normal RNA synthesis as described above and in Usman et al., (1987 J. Am. Chem. Soc., 109, 7845), Scaringe et al., (1990 Nucleic Acids Res., 18, 5433) and Wincott et al., supra, and made use of common nucleic acid protecting and coupling groups, such as dimethoxytrityl at the 5′-end, and phosphoramidites at the 3′-end. The average stepwise coupling yields were typically >98%.

Nucleic acid molecules are also synthesized from DNA templates using bacteriophage T7 RNA polymerase (Milligan and Uhlenbeck, 1989, Methods Enzymol. 180, 51). Nucleic acid molecules of the invention are purified by gel electrophoresis using general methods or are purified by high pressure liquid chromatography (HPLC; See Wincott et al., supra; the totality of which is hereby incorporated herein by reference) and are resuspended in water. Examples of sequences of chemically synthesized enzymatic nucleic acid molecules are shown in Formula I (SEQ ID NO: 13) and in Pavco et al., U.S. patent application Ser. No. 09/870,161.

### EXAMPLE 4

### Enzymatic Nucleic Acid Molecule Cleavage of VEGFR1 and/or VEGFR2 RNA Target In Vitro

Enzymatic nucleic acid molecules targeted to the human VEGFR1 and/or VEGFR2 RNA are designed and synthesized as described above. These enzymatic nucleic acid molecules can be tested for cleavage activity in vitro, for example, using the following procedure. The target sequences and the nucleotide location within the VEGFR1 and/or VEGFR2 RNA are described in Pavco et al., U.S. patent application Ser. No. 09/870,161.

Cleavage Reactions. Full-length or partially full-length, internally-labeled target RNA for enzymatic nucleic acid molecule cleavage assay is prepared by in vitro transcription in the presence of [a-³²P] CTP, passed over a G 50 Sephadex column by spin chromatography and used as substrate RNA without further purification. Alternately, substrates are 5′-³²P-end labeled using T4 polynucleotide kinase enzyme. Assays are performed by pre-warming a 2× concentration of purified enzymatic nucleic acid molecule in enzymatic nucleic acid molecule cleavage buffer (50 mM Tris-HCl, pH 7.5 at 37° C., 10 mM MgCl₂) and the cleavage reaction was initiated by adding the 2× enzymatic nucleic acid molecule mix to an equal volume of substrate RNA (maximum of 1-5 nM) that was also pre-warmed in cleavage buffer. As an initial screen, assays are carried out for 1 hour at 37° C. using a final concentration of either 40 nM or 1 mM enzymatic nucleic acid molecule, i.e., enzymatic nucleic acid molecule excess. The reaction is quenched by the addition of an equal volume of 95% formamide, 20 mM EDTA, 0.05% bromophenol blue and 0.05% xylene cyanol after which the sample is heated to 95° C. for 2 minutes, quick chilled and loaded onto a denaturing polyacrylamide gel. Substrate RNA and the specific RNA cleavage products generated by enzymatic nucleic acid molecule cleavage are visualized on an autoradiograph of the gel. The percentage of cleavage is determined by Phosphor Imager® quantitation of bands representing the intact substrate and the cleavage products.

### Example 5

### Phase I/II Study of Repetitive Dosing of ANGIOZYME™ Targeting the FLT-1 Receptor of VEGF

A ribozyme therapeutic agent ANGIOZYMETM (SEQ ID NO: 13), was assessed by daily subcutaneous (sc) administration in a phase I/II trial for 31 patients with refractory solid tumors. Demographic information relating to patients enrolled in the study are shown in Table III. The primary study endpoint was to determine the safety and maximum tolerated dose of ANGIOZYMETM. Secondary endpoints assessed ANGIOZYMETM pharmacokinetics and clinical response. Patients were treated at the following doses: 3 patients received doses of 10 mg/m²/day, 4 patients received 30 mg/m²/day, 20 patients received 100 mg/m²/day, and 4 patients received 300 mg/m²/day. All but one patient were dosed for a minimum of 29 consecutive days with 24-hour pharmacokinetic analyses on Day 1 and 29. Clinical response was assessed monthly.

Results

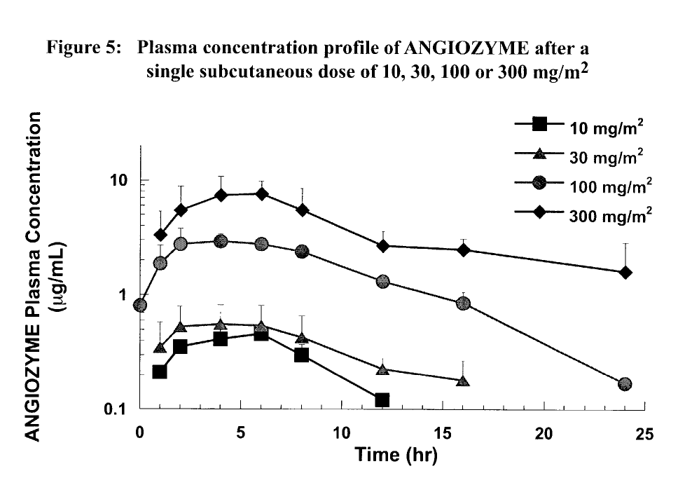

The data from 20 patients indicated that ANGIOZYME™ was well tolerated, with no systemic adverse events. FIG. 5 shows the plasma concentration profile of ANGIOZYME™ after a single SC (sub-cutaneous) dose of 10, 30, 100, or 300 mg/m². The pharmacokinetic parameters of ANGIOZYME™ after SC bolus administration are outlined in Table IV. An MTD (maximum tolerated dose) could not be established. One patient in the 300 mg/m²/d group experienced a grade 3 injection site reaction. Patients in the other groups experienced intermittent grade 1 and grade 2 injection site reactions with erythema and induration. No systemic or laboratory toxicities were observed. Pharmacokinetic analyses demonstrated dose-dependent plasma concentrations with good bioavailability (70-90%), t1/2=209-384 min, and no accumulation after repeated doses. To date, 17/28 (61%) of evaluable patients have had stable disease for periods of one to six months and two patients (nasopharyngeal squamous cell carcinoma and melanoma) had minor clinical responses. The patient with nasopharyngeal carcinoma demonstrated central tumor necrosis as indicated by MRI. The longest period of treatment thus far has been 8 months for two patients at 100 mg/m²/d (breast, peritoneal mesothelioma).

### EXAMPLE 6

### Down-Regulation of VEGFR1 Gene Expression to Treat Gynecologic Neovascularization Dependent Conditions

One patient in the Phase I/II trial described in Example 5 was menstruating prior to enrollment in the ANGIOZYME™ monotherapy trial. After 1-2 months on trial, the patient's menstrual cycles ceased. The patient remained on trial for approximately 11 months and did not menstruate. The patient then went off the trial for about 4 months and the menstrual cycles resumed. Re-enrollment in the ANGIOZYME™ trial resulted in the patient's menstrual cycle stopping again. This clinical observation suggests that ANGIOZYME™ is interfering with the patient's menstrual cycle, perhaps by inhibiting neovascularization of uterine tissue. This data also suggests that ANGIOZYME™ has a direct effect on the endometrial tissue or an effect on LH/FSH stimulation. These results suggest the treatment or control, using ANGIOZYME™ (SEQ ID NO: 13) and/or other nucleic acid molecules of the instant invention, of various clinical targets and/or processes associated with female reproduction and gynecologic neovascularization, such as endometriosis, birth control, gynecologic bleeding disorders, irregular menstrual cycles, ovulation, premenstrual syndrome (PMS), menopausal dysfunction, endometrial carcinoma or any other condition associated with the expression of VEGFR1 and/or VEGFR2 VEGF receptors.

Indications

Various studies indicate that VEGF is directly implicated in endometriosis. In one study, VEGF concentrations measured by ELISA in peritoneal fluid were found to be significantly higher in women with endometriosis than in women without endometriosis (24.1±15 ng/ml vs 13.3±7.2 ng/ml in normals). In patients with endometriosis, higher concentrations of VEGF were detected in the proliferative phase of the menstrual cycle (33±13 ng/ml) compared to the secretory phase (10.7±5 ng/ml). The cyclic variation was not noted in fluid from normal patients (McLaren et al., 1996, Human Reprod. 11, 220-223). In another study, women with moderate to severe endometriosis had significantly higher concentrations of peritoneal fluid VEGF than women without endometriosis. There was a positive correlation between the severity of endometriosis and the concentration of VEGF in peritoneal fluid. In human endometrial biopsies, VEGF expression increased relative to the early proliferative phase approximately 1.6-, 2-, and 3.6-fold in midproliferative, late proliferative, and secretory endometrium (Shifren et al., 1996, J. Clin. Endocrinol. Metab. 81, 3112-3118).

In a third study, VEGF-positive staining of human ectopic endometrium was shown to be localized to macrophages (double immunofluorescent staining with CD14 marker). Peritoneal fluid macrophages demonstrated VEGF staining in women with and without endometriosis. However, increased activation of macrophages (acid phosphatatse activity) was demonstrated in fluid from women with endometriosis compared with controls. Peritoneal fluid macrophage conditioned media from patients with endometriosis resulted in significantly increased cell proliferation ([³H] thymidine incorporation) in HUVEC cells compared to controls. The percentage of peritoneal fluid macrophages with VEGFR2 mRNA was higher during the secretory phase, and significantly higher in fluid from women with endometriosis (80±15%) compared with controls (32±20%). Flt-mRNA was detected in peritoneal fluid macrophages from women with and without endometriosis, but there was no difference between the groups or any evidence of cyclic dependence (McLaren et al., 1996, J. Clin. Invest. 98, 482-489).

In the early proliferative phase of the menstrual cycle, VEGF has been found to be expressed in secretory columnar epithelium (estrogen-responsive) lining both the oviducts and the uterus in female mice. During the secretory phase, VEGF expression was shown to have shifted to the underlying stroma composing the functional endometrium. In addition to examining the endometium, neovascularization of ovarian follicles and the corpus luteum, as well as angiogenesis in embryonic implantation sites have been analyzed. For these processes, VEGF was expressed in spatial and temporal proximity to forming vasculature (Shweiki et al., 1993, J. Clin. Invest. 91, 2235-2243).

The present body of knowledge in VEGFR1 and/or VEGFR2 research indicates the need for methods to assay VEGFR1 and/or VEGFR2 activity and for compounds that can regulate VEGFR1 and/or VEGFR2 expression for research, diagnostic, and therapeutic use. As described herein, the nucleic acid molecules of the present invention can be used in assays to diagnose disease state related of VEGF, VEGFR1 and/or VEGFR2 levels. In addition, the nucleic acid molecules can be used to treat disease state related to VEGF and/or VEGFr, such as VEGFR1 and/or VEGFR2 levels.

Particular processes, diseases, or conditions that can be associated with VEGFR1 and/or VEGFR2 levels include, but are not limited to, gynecologic neovascularization, such as endometriosis, endometrial carcinoma, gynecologic bleeding disorders, irregular menstrual cycles, ovulation, premenstrual syndrome (PMS), menopausal dysfunction and any other diseases or conditions that are related to or will respond to the levels of VEGF and/or VEGFr, such as VEGFRI and/or VEGFR2 in a cell or tissue, alone or in combination with other therapies The use of GNRH (gonadotropin releasing hormone) agonists, Lupron Depot (Leuprolide Acetate), Synarel (naferalin acetate), Zolodex (goserelin acetate), Suprefact (buserelin acetate), Danazol, or oral contraceptives including but not limited to Depo-Provera or Provera (medroxyprogesterone acetate), or any other estrogen/progesterone contraceptive, are all non-limiting examples of a methods that can be combined with or used in conjunction with the nucleic acid molecules of the instant invention. Various chemotherapies can be readily combined with nucleic acid molecules of the invention for the treatment of endometrial carcinoma. Common chemotherapies that can be combined with nucleic acid molecules of the instant invention include various combinations of cytotoxic drugs to kill the cancer cells. These drugs include but are not limited to paclitaxel (Taxol), docetaxel, cisplatin, methotrexate, cyclophosphamide, doxorubin, fluorouracil carboplatin, edatrexate, gemcitabine, vinorelbine etc. Those skilled in the art will recognize that other drug compounds and therapies can be readily combined with the nucleic acid molecules of the instant invention and are hence within the scope of the instant invention.

Animal Models

Surgically induced models of endometriosis have been developed in rats, mice, and rabbits. Non-human primates demonstrate spontaneous endometriosis, but surgical induction can also be used. In addition to the surgical technique, cycle monitoring can be performed by daily vaginal cytology in primates. For all of the surgically induced models of endometriosis, the following general procedure is used. An initial laparotomy is performed to implant tissue from a donor animal. A portion of one uterine horn (or one complete horn in the case of mice) is removed. The endometrium of this piece of uterus is separated from the myometrium and cut into small segments (4-10 mm2). Segments (approximately 3) are sutured to various locations within the abdominal cavity (peritoneum, intestinal mesentery vessels, uterus, broad ligament). Cummings and Metcalf (1996) attached whole segments of mouse uterus without separating the endometrium from the myometrium. Implants are allowed to grow for 3-6 weeks. A second laparotomy is sometimes performed to verify development of endometriosis-like foci (vascularization and cysts filled with clear fluid). This second laparotomy was done in the studies by Quereda et al., (1996) and Stoeckemann et al., (1995). After 3-6 weeks post-surgery and/or following visualization of endometriosis, drug treatment is initiated and continued for a prescribed period of time. At the termination of these studies, animals are euthanized. Endpoints include, but are not limited to, changes in the surface area of the implants and tissue mass of the ectopic endometrial implants (see for example Brogniez et al., 1995, Human Reprod. 10, 927-931; Cummings et al., 1996, Tox. Appl. Pharm. 138, 131-139; Cummings and Metcalf, 1996, Proc. Soc. Exp. Biol. Med. 212, 332-337; D'Hooghe et al., 1996, Fertility and Sterility. 66, 809-813; Quereda et al., 1996, Eur. J. Obstet. Gynecol. Rep. Biol. 67, 35-40; and Stoeckemann et al., 1995, Human Reprod. 10, 3264-3271).

Diagnostic Uses

The nucleic acid molecules of this invention can be used as diagnostic tools to examine genetic drift and mutations within diseased cells or to detect the presence of VEGF and/or VEGFr, such as VEGFR1 and/or VEGFR2 RNA in a cell. For example, the close relationship between enzymatic nucleic acid molecule activity and the structure of the target RNA allows the detection of mutations in any region of the molecule which alters the base-pairing and three-dimensional structure of the target RNA. By using multiple enzymatic nucleic acid molecules described in this invention, one can map nucleotide changes which are important to RNA structure and function in vitro, as well as in cells and tissues. Cleavage of target RNAs with enzymatic nucleic acid molecules can be used to inhibit gene expression and define the role (essentially) of specified gene products in the progression of disease. In this manner, other genetic targets can be defined as important mediators of the disease. These experiments can lead to better treatment of the disease progression by affording the possibility of combinational therapies (e.g., multiple enzymatic nucleic acid molecules targeted to different genes, enzymatic nucleic acid molecules coupled with known small molecule inhibitors, or intermittent treatment with combinations of enzymatic nucleic acid molecules and/or other chemical or biological molecules). Other in vitro uses of enzymatic nucleic acid molecules of this invention are well known in the art, and include detection of the presence of mRNAs associated with VEGF, VEGFRI and/or VEGFR2-related condition. Such RNA is detected by determining the presence of a cleavage product after treatment with an enzymatic nucleic acid molecule using standard methodology.

In a specific example, enzymatic nucleic acid molecules which cleave only wild-type or mutant forms of the target RNA are used for the assay. The first enzymatic nucleic acid molecule is used to identify wild-type RNA present in the sample and the second enzymatic nucleic acid molecule is used to identify mutant RNA in the sample. As reaction controls, synthetic substrates of both wild-type and mutant RNA are cleaved by both enzymatic nucleic acid molecules to demonstrate the relative enzymatic nucleic acid molecule efficiencies in the reactions and the absence of cleavage of the “non-targeted” RNA species. The cleavage products from the synthetic substrates also serve to generate size markers for the analysis of wild-type and mutant RNAs in the sample population. Thus each analysis requires two enzymatic nucleic acid molecules, two substrates and one unknown sample which is combined into six reactions. The presence of cleavage products is determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., VEGFR1 and/or VEGFR2) is adequate to establish risk. If probes of comparable specific activity are used for both transcripts, then a qualitative comparison of RNA levels will be adequate and will decrease the cost of the initial diagnosis. Higher mutant form to wild-type ratios are correlated with higher risk whether RNA levels are compared qualitatively or quantitatively. The use of enzymatic nucleic acid molecules in diagnostic applications contemplated by the instant invention is described, for example, in Usman et al., U.S. patent application Ser. No. 09/877,526, George et al, U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No.

WO 00/24931, Breaker et al., International PCT Publication Nos. WO 00/26226 and 98/27104, and Sullenger et al., U.S. patent application Ser. No. 09/205,520.

Additional Uses

Potential uses of sequence-specific enzymatic nucleic acid molecules of the instant invention can have many of the same applications for the study of RNA that DNA restriction endonucleases have for the study of DNA (Nathans et al., 1975 Ann. Rev. Biochem. 44:273). For example, the pattern of restriction fragments can be used to establish sequence relationships between two related RNAs, and large RNAs can be specifically cleaved to fragments of a size more useful for study. The ability to engineer sequence specificity of the enzymatic nucleic acid molecule is ideal for cleavage of RNAs of unknown sequence. Applicant has described the use of nucleic acid molecules to down-regulate gene expression of target genes in bacterial, microbial, fungal, viral, and eukaryotic systems including plant, or mammalian cells.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the following claims.

### TABLE I

Characteristics of Naturally Occurring Ribozymes

Group I Introns

Size: ˜150 to >1000 nucleotides.

Requires a U in the target sequence immediately 5′ of the cleavage site.

Binds 4-6 nucleotides at the 5′-side of the cleavage site.

Reaction mechanism: attack by the 3′-OH of guanosine to generate cleavage products with 3′-OH and 5′-guanosine.

Additional protein cofactors required in some cases to help folding and maintenance of the active structure.

Over 300 known members of this class. Found as an intervening sequence in Tetrahymena thermophila rRNA, fungal mitochondria, chloroplasts, phage T4, blue-green algae, and others.

Major structural features largely established through phylogenetic comparisons, mutagenesis, and biochemical studies [ⁱ,ⁱⁱ].

Complete kinetic framework established for one ribozyme [ⁱⁱⁱ,^{iv},^{v},^{vi}].

Studies of ribozyme folding and substrate docking underway [^{vii},^{viii},^{ix}].

Chemical modification investigation of important residues well established [^{x},^{xi}].

The small (4-6 nt) binding site may make this ribozyme too non-specific for targeted RNA cleavage, however, the Tetrahymena group I intron has been used to repair a “defective” β-galactosidase message by the ligation of new β-galactosidase sequences onto the defective message [^{xii}].

RNAse P RNA (M1 RNA)

Size: ˜290 to 400 nucleotides.

RNA portion of a ubiquitous ribonucleoprotein enzyme.

Cleaves tRNA precursors to form mature tRNA [^{xiii}].

Reaction mechanism: possible attack by M²⁺-OH to generate cleavage products with 3′-OH and 5′-phosphate.

RNAse P is found throughout the prokaryotes and eukaryotes. The RNA subunit has been sequenced from bacteria, yeast, rodents, and primates.

Recruitment of endogenous RNAse P for therapeutic applications is possible through hybridization of an External Guide Sequence (EGS) to the target RNA [^{xiv},^{xv}]

Important phosphate and 2′ OH contacts recently identified [^{xvi},^{xvii}]

Group II Introns

Size: >1000 nucleotides.

Trans cleavage of target RNAs recently demonstrated [^{xviii},^{xix}].

Sequence requirements not fully determined.

Reaction mechanism: 2′-OH of an internal adenosine generates cleavage products with 3′-OH and a “lariat” RNA containing a 3′-5′ and a 2′-5′ branch point.

Only natural ribozyme with demonstrated participation in DNA cleavage [^{xx},^{xxi}] in addition to RNA cleavage and ligation.

Major structural features largely established through phylogenetic comparisons [^{xxii}].

Important 2′ OH contacts beginning to be identified [^{xxii}]

Kinetic framework under development [^{xxiv}]

Neurospora VS RNA

Size: ˜144 nucleotides.

Trans cleavage of hairpin target RNAs recently demonstrated [^{xxv}].

Sequence requirements not fully determined.

Reaction mechanism: attack by 2′-OH 5′ to the scissile bond to generate cleavage products with 2′,3′-cyclic phosphate and 5′-OH ends.

Binding sites and structural requirements not fully determined.

Only 1 known member of this class. Found in Neurospora VS RNA.

Hammerhead Ribozyme

(See Text for References)

Size: ˜13 to 40 nucleotides.

Requires the target sequence UH immediately 5′ of the cleavage site.

Binds a variable number nucleotides on both sides of the cleavage site.

Reaction mechanism: attack by 2′-OH 5′ to the scissile bond to generate cleavage products with 2′,3′-cyclic phosphate and 5′-OH ends.

14 known members of this class. Found in a number of plant pathogens (virusoids) that use RNA as the infectious agent.

Essential structural features largely defined, including 2 crystal structures [^{xxvi},^{xxvii}]

Minimal ligation activity demonstrated (for engineering through in vitro selection) [^{xxviii}]

Complete kinetic framework established for two or more ribozymes [^{xxix}].

Chemical modification investigation of important residues well established [^{xxx}].

Hairpin Ribozyme

Size: ˜50 nucleotides.

Requires the target sequence GUC immediately 3′ of the cleavage site.

Binds 4-6 nucleotides at the 5′-side of the cleavage site and a variable number to the 3′-side of the cleavage site.

Reaction mechanism: attack by 2′-OH 5′ to the scissile bond to generate cleavage products with 2′,3′-cyclic phosphate and 5′-OH ends.

3 known members of this class. Found in three plant pathogen (satellite RNAs of the tobacco ringspot virus, arabis mosaic virus and chicory yellow mottle virus) which uses RNA as the infectious agent.

Essential structural features largely defined [^{xxxi},^{xxxii},^{xxxiii},^{xxxiv}]

Ligation activity (in addition to cleavage activity) makes ribozyme amenable to engineering through in vitro selection [^{xxxv}]

Complete kinetic framework established for one ribozyme [^{xxxvi}].

Chemical modification investigation of important residues begun [^{xxxvii},^{xxxviii}].

Hepatitis Delta Virus (HDV) Ribozyme

Size: ˜60 nucleotides.

Trans cleavage of target RNAs demonstrated [^{xxxix}].

Binding sites and structural requirements not fully determined, although no sequences 5′ of cleavage site are required. Folded ribozyme contains a pseudoknot structure [^{xl}].

Reaction mechanism: attack by 2′-OH 5′ to the scissile bond to generate cleavage products with 2′,3′-cyclic phosphate and 5′-OH ends.

Only 2 known members of this class. Found in human HDV.

Circular form of HDV is active and shows increased nuclease stability [^{xli}]

ⁱ. Michel, Francois; Westhof, Eric. Slippery substrates. Nat. Struct. Biol. (1994), 1(1), 5-7.

ⁱⁱ. Lisacek, Frederique; Diaz, Yolande; Michel, Francois. Automatic identification of group I intron cores in genomic DNA sequences. J. Mol. Biol. (1994), 235(4), 1206-17.

ⁱⁱⁱ. Herschlag, Daniel; Cech, Thomas R. Catalysis of RNA cleavage by the Tetrahymena thermophila ribozyme. 1. Kinetic description of the reaction of an RNA substrate complementary to the active site. Biochemistry (1990), 29(44), 10159-71.

^{iv}. Herschlag, Daniel; Cech, Thomas R. Catalysis of RNA cleavage by the Tetrahymena thermophila ribozyme. 2. Kinetic description of the reaction of an RNA substrate that forms a mismatch at the active site. Biochemistry (1990), 29(44), 10172-80.

^{v}. Knitt, Deborah S.; Herschlag, Daniel. pH Dependencies of the Tetrahymena Ribozyme Reveal an Unconventional Origin of an Apparent pKa. Biochemistry (1996), 35(5), 1560-70.

^{vi}. Bevilacqua, Philip C.; Sugimoto, Naoki; Turner, Douglas H. A mechanistic framework for the second step of splicing catalyzed by the Tetrahymena ribozyme. Biochemistry (1996), 35(2), 648-58.

^{vii}. Li, Yi; Bevilacqua, Philip C.; Mathews, David; Turner, Douglas H. Thermodynamic and activation parameters for binding of a pyrene-labeled substrate by the Tetrahymena ribozyme: docking is not diffusion-controlled and is driven by a favorable entropy change. Biochemistry (1995), 34(44), 14394-9.

^{viii}. Banerjee, Aloke Raj; Turner, Douglas H. The time dependence of chemical modification reveals slow steps in the folding of a group I ribozyme. Biochemistry (1995), 34(19), 6504-12.

^{ix}. Zarrinkar, Patrick P.; Williamson, James R. The P9.1-P9.2 peripheral extension helps guide folding of the Tetrahymena ribozyme. Nucleic Acids Res. (1996), 24(5), 854-8.

^{x}. Strobel, Scott A.; Cech, Thomas R. Minor groove recognition of the conserved G.cntdot.U pair at the Tetrahymena ribozyme reaction site. Science (Washington, D. C.) (1995), 267(5198), 675-9.

^{xi}. Strobel, Scott A.; Cech, Thomas R. Exocyclic Amine of the Conserved G.cntdot.U Pair at the Cleavage Site of the Tetrahymena Ribozyme Contributes to 5′-Splice Site Selection and Transition State Stabilization. Biochemistry (1996), 35(4), 1201-11.

^{xii}. Sullenger, Bruce A.; Cech, Thomas R. Ribozyme-mediated repair of defective mRNA by targeted trans-splicing. Nature (London) (1994), 371(6498), 619-22.

^{xiii}. Robertson, H. D.; Altman, S.; Smith, J. D. J. Biol. Chem., 247 5243-5251 (1972).

^{xiv}. Forster, Anthony C.; Altman, Sidney. External guide sequences for an RNA enzyme. Science (Washington, D. C., 1883-) (1990), 249(4970), 783-6.

^{xv}. Yuan, Y.; Hwang, E. S.; Altman, S. Targeted cleavage of mRNA by human RNase P. Proc. Natl. Acad. Sci. USA (1992) 89, 8006-10.

^{xvi}. Harris, Michael E.; Pace, Norman R. Identification of phosphates involved in catalysis by the ribozyme RNase P RNA. RNA (1995), 1(2), 210-18.

^{xvii}. Pan, Tao; Loria, Andrew; Zhong, Kun. Probing of tertiary interactions in RNA: 2′-hydroxyl-base contacts between the RNase P RNA and pre-tRNA. Proc. Natl. Acad. Sci. U.S. A. (1995), 92(26), 12510-14.

^{xviii}. Pyle, Anna Marie; Green, Justin B. Building a Kinetic Framework for Group II Intron Ribozyme Activity: Quantitation of Interdomain Binding and Reaction Rate. Biochemistry (1994), 33(9), 2716-25.

^{xix}. Michels, William J. Jr.; Pyle, Anna Marie. Conversion of a Group II Intron into a New Multiple-Turnover Ribozyme that Selectively Cleaves Oligonucleotides: Elucidation of Reaction Mechanism and Structure/Function Relationships. Biochemistry (1995), 34(9), 2965-77.

^{xx}. Zimmerly, Steven; Guo, Huatao; Eskes, Robert; Yang, Jian; Perlman, Philip S.; Lambowitz, Alan M. A group II intron RNA is a catalytic component of a DNA endonuclease involved in intron mobility. Cell (Cambridge, Mass.) (1995), 83(4), 529-38.

^{xxi}. Griffin, Edmund A., Jr.; Qin, Zhifeng; Michels, Williams J., Jr.; Pyle, Anna Marie. Group II intron ribozymes that cleave DNA and RNA linkages with similar efficiency, and lack contacts with substrate 2′-hydroxyl groups. Chem. Biol. (1995), 2(11), 761-70.

^{xxii}. Michel, Francois; Ferat, Jean Luc. Structure and activities of group II introns. Annu. Rev. Biochem. (1995), 64, 435-61.

^{xxiii}. Abramovitz, Dana L.; Friedman, Richard A.; Pyle, Anna Marie. Catalytic role of 2′-hydroxyl groups within a group II intron active site. Science (Washington, D. C.) (1996), 271(5254), 1410-13.

^{xxiv}. Daniels, Danette L.; Michels, William J., Jr.; Pyle, Anna Marie. Two competing pathways for self-splicing by group II introns: a quantitative analysis of in vitro reaction rates and products. J. Mol. Biol. (1996), 256(1), 31-49.

^{xxv}. Guo, Hans C. T.; Collins, Richard A. Efficient trans-cleavage of a stem-loop RNA substrate by a ribozyme derived from Neurospora VS RNA. EMBO J. (1995), 14(2), 368-76.

^{xxvi}. Scott, W. G., Finch, J. T., Aaron, K. The crystal structure of an all RNA hammerhead ribozyme:Aproposed mechanism for RNA catalytic cleavage. Cell, (1995), 81, 991-1002.

^{xxvii}. McKay, Structure and function of the hammerhead ribozyme: an unfinished story. RNA, (1996), 2, 395-403.

^{xxviii}. Long, D., Uhlenbeck, O., Hertel, K. Ligation with hammerhead ribozymes. U.S. Pat. No. 5,633,133.

^{xxix}. Hertel, K. J., Herschlag, D., Uhlenbeck, O. A kinetic and thermodynamic framework for the hammerhead ribozyme reaction. Biochemistry, (1994) 33, 3374-3385.Beigelman, L., et al., Chemical modifications of hammerhead ribozymes. J. Biol. Chem., (1995) 270, 25702-25708.

^{xxx}. Beigelman, L., et al., Chemical modifications of hammerhead ribozymes. J. Biol. Chem., (1995) 270, 25702-25708.

^{xxxi}. Hampel, Arnold; Tritz, Richard; Hicks, Margaret; Cruz, Phillip. ‘Hairpin’ catalytic RNA model: evidence for helixes and sequence requirement for substrate RNA. Nucleic Acids Res. (1990), 18(2), 299-304.

^{xxxii}. Chowrira, Bharat M.; Berzal-Herranz, Alfredo; Burke, John M. Novel guanosine requirement for catalysis by the hairpin ribozyme. Nature (London) (1991), 354(6351), 320-2.

^{xxxiii}. Berzal-Herranz, Alfredo; Joseph, Simpson; Chowrira, Bharat M.; Butcher, Samuel E.; Burke, John M. Essential nucleotide sequences and secondary structure elements of the hairpin ribozyme. EMBO J. (1993), 12(6), 2567-73.

^{xxxiv}. Joseph, Simpson; Berzal-Herranz, Alfredo; Chowrira, Bharat M.; Butcher, Samuel E. Substrate selection rules for the hairpin ribozyme determined by in vitro selection, mutation, and analysis of mismatched substrates. Genes Dev. (1993), 7(1), 130-8.

^{xxxv}. Berzal-Herranz, Alfredo; Joseph, Simpson; Burke, John M. In vitro selection of active hairpin ribozymes by sequential RNA-catalyzed cleavage and ligation reactions. Genes Dev. (1992), 6(1), 129-34.

^{xxxvi}. Hegg, Lisa A.; Fedor, Martha J. Kinetics and Thermodynamics of Intermolecular Catalysis by Hairpin Ribozymes. Biochemistry (1995), 34(48), 15813-28.

^{xxxvii}. Grasby, Jane A.; Mersmann, Karin; Singh, Mohinder; Gait, Michael J. Purine Functional Groups in Essential Residues of the Hairpin Ribozyme Required for Catalytic Cleavage of RNA. Biochemistry (1995), 34(12), 4068-76.

^{xxxviii}. Schmidt, Sabine; Beigelman, Leonid; Karpeisky, Alexander; Usman, Nassim; Sorensen, Urik S.; Gait, Michael J. Base and sugar requirements for RNA cleavage of essential nucleoside residues in internal loop B of the hairpin ribozyme: implications for secondary structure. Nucleic Acids Res. (1996), 24(4), 573-81.

^{xxxix}. Perrotta, Anne T.; Been, Michael D. Cleavage of oligoribonucleotides by a ribozyme derived from the hepatitis delta. virus RNA sequence. Biochemistry (1992), 31(1), 16-21.

^{xl}. Perrotta, Anne T.; Been, Michael D. A pseudoknot-like structure required for efficient self-cleavage of hepatitis delta virus RNA. Nature (London) (1991), 350(6317), 434-6.

^{xli}. Puttaraju, M.; Perrotta, Anne T.; Been, Michael D. A circular trans-acting hepatitis delta virus ribozyme. Nucleic Acids Res. (1993), 21(18), 4253-8.

TABLE II
A. 2.5 μmol Synthesis Cycle ABI 394 Instrument

| Reagent | Equivalents | Amount | | Wait Time* DNA | Wait Time* 2'-O-methyl | | Wait Time* RNA | |
|---|---|---|---|---|---|---|---|---|
| Phosphoramidites | 6.5 | 163 | μL | 45 sec | 2.5 | min | 7.5 | min |
| S-Ethyl Tetrazole | 23.8 | 238 | μL | 45 sec | 2.5 | min | 7.5 | min |
| Acetic Anhydride | 100 | 233 | μL | 5 sec | 5 | sec | 5 | sec |
| N-Methyl | 186 | 233 | μL | 5 sec | 5 | sec | 5 | sec |
| Imidazole | | | | | | | | |
| TCA | 176 | 2.3 | mL | 21 sec | 21 | sec | 21 | sec |
| Iodine | 11.2 | 1.7 | mL | 45 sec | 45 | sec | 45 | sec |
| Beaucage | 12.9 | 645 | μL | 100 sec | 300 | sec | 300 | sec |
| Acetonitrile | NA | 6.67 | mL | NA | NA | | NA | |

B. 0.2 μmol Synthesis Cycle ABI 394 Instrument

| Reagent | Equivalents | Amount | | Wait Time* DNA | Wait Time* 2'-O-methyl | | Wait Time* RNA | |
|---|---|---|---|---|---|---|---|---|
| Phosphoramidites | 15 | 31 | μL | 45 sec | 233 | sec | 465 | sec |
| S-Ethyl Tetrazole | 38.7 | 31 | μL | 45 sec | 233 | min | 465 | sec |
| Acetic Anhydride | 655 | 124 | μL | 5 sec | 5 | sec | 5 | sec |
| N-Methyl | 1245 | 124 | μL | 5 sec | 5 | sec | 5 | sec |
| Imidazole | | | | | | | | |
| TCA | 700 | 732 | μL | 10 sec | 10 | sec | 10 | sec |
| Iodine | 20.6 | 244 | μL | 15 sec | 15 | sec | 15 | sec |
| Beaucage | 7.7 | 232 | μL | 100 sec | 300 | sec | 300 | sec |
| Acetonitrile | NA | 2.64 | mL | NA | NA | | NA | |

C. 0.2 μmol Synthesis Cycle 96 well Instrument

| | Equivalents: DNA/ | Amount: DNA/2'-O- | | | Wait Time* 2'-O- | | Wait Time* |
|---|---|---|---|---|---|---|---|
| Reagent | 2'-O-methyl/Ribo | methyl/Ribo | | Wait Time* DNA | methyl | | Ribo |
| Phosphoramidites | 22/33/66 | 40/60/120 | μL | 60 sec | 180 | sec | 360 sec |
| S-Ethyl Tetrazole | 70/105/210 | 40/60/120 | μL | 60 sec | 180 | min | 360 sec |
| Acetic Anhydride | 265/265/265 | 50/50/50 | μL | 10 sec | 10 | sec | 10 sec |
| N-Methyl | 502/502/502 | 50/50/50 | μL | 10 sec | 10 | sec | 10 sec |
| Imidazole | | | | | | | |
| TCA | 238/475/475 | 250/500/500 | μL | 15 sec | 15 | sec | 15 sec |
| Iodine | 6.8/6.8/6.8 | 80/80/80 | μL | 30 sec | 30 | sec | 30 sec |
| Beaucage | 34/51/51 | 80/120/120 | | 100 sec | 200 | sec | 200 sec |
| Acetonitrile | NA | 1150/1150/1150 | μL | NA | NA | | NA |

*Wait time does not include contact time during delivery.

TABLE III
Patient Demographics

| Dose cohort | | | | | |
|---|---|---|---|---|---|
| (mg/m²) | Pt# | Age | Sex | Diagnosis | Doses |
| 10 | 1001 | 49 | F | NSC Lung | 29 |
| 10 | 1002 | 65 | F | liposarcoma | 120 |
| 10 | 1003 | 49 | M | nasopharyngeal CA | 109 |
| 30 | 1004 | 35 | M | non-small cell lung | 1 |
| 30 | 1005 | 45 | F | melanoma (ocular) | 113 |
| 30 | 1006 | 57 | M | colon | 199 |
| 30 | 1007 | 39 | F | epitheliod | 198 |
| | | | | hemangioendothelioma | |
| 100 | 1008 | 52 | M | adrenal CA | 57 |
| 100 | 1009 | 44 | F | breast | 35 |
| 100 | 1010 | 62 | F | renal | 134 |
| 300 | 1011 | 24 | F | melanoma | 31 |
| 300 | 1012 | 57 | M | renal cell | 178 |
| 300 | 1013 | 53 | M | nasopharyngeal SCCA | 29 |
| 300 | 1014 | 64 | F | peritoneal mesothelioma | 324 |
| 100 | 1015 | 65 | M | melanoma | 140 |
| 100 | 1016 | 77 | F | breast | 265 |
| 100 | 1017 | | F | melanoma | 35 |
| 100 | 1018 | 26 | F | melanoma | 7 |
| 100 | 1019 | 69 | F | endometrial sarcoma | 500 |
| 100 | 1020 | 65 | M | carcinoid | 124 |
| 100 | 1021 | 59 | M | gallbladder adeno | 34 |
| | | | | carcinoma | |
| 100 | 1022 | 43 | M | colorectal | 8 |
| 100 | 1023 | 78 | F | breast | 50 |
| 100 | 1024 | 40 | F | parotid adenocarcinoma | 285 |
| 100 | 1025 | 52 | F | breast | 71 |
| 100 | 1026 | 39 | F | breast | 34 |
| 100 | 1027 | 55 | F | breast | 36 |
| 100 | 1028 | 52 | M | melanoma | 29 |
| 100 | 1029 | 38 | M | pancreatic | 36 |
| 100 | 1030 | 83 | M | melanoma | 41 |
| 100 | 1031 | 50 | M | medullary thyroid | 108 |

One patient taken off study due to progressive disease. Allowed to resume ANGIOZYME on a compassionate basis.

As of Sep. 1, 2001, all patients were off study. (Although one patient resumed treatment per above note)

TABLE IV
Pharmacokinetic parameters of ANGIOZYME after bolus subcutaneous administration.

| | | 10 mg/m² | | 30 mg/M² | | 100 mg/m² | | 300 mg/m² | |
|---|---|---|---|---|---|---|---|---|---|
| | | Mean | SD | Mean | SD | Mean | SD | Mean | SD |
| Day 1 | Cmax (ug/mL) | 0.43 | 0.07 | 0.62 | 0.28 | 3.17 | 0.69 | 8.91 | 2.93 |
| | AUCt (ug*hr/mL) | 2.60 | 1.43 | 6.04 | 2.70 | 34.14 | 2.28 | 89.87 | 21.68 |
| | AUCinf (ug*hr/mL) | 4.40 | 0.06 | 7.99 | 1.66 | 37.51 | 1.91 | 101.57 | 13.47 |
| | t(1/2) (hr) | 3.62 | 0.79 | 7.32 | 6.94 | 4.58 | 0.02 | 9.26 | 6.20 |
| | CL/F (L/hr/m²) | 2.24 | 0.08 | 3.73 | 0.92 | 2.96 | 0.61 | 2.99 | 0.43 |
| Day 29 | Cmax (ug/mL) | 0.35 | 0.19 | 1.17 | 0.53 | 3.23 | 0.35 | 8.93 | 6.71 |
| | AUCt (ug*hr/mL) | 2.11 | 1.31 | 7.29 | 1.16 | 31.87 | 1.91 | 119.42 | 65.84 |
| | AUCinf (ug*hr/mL) | 3.38 | 1.31 | 8.54 | 2.46 | 33.61 | 2.16 | 132.73 | 67.82 |
| | t(1/2) (hr) | 4.49 | 1.60 | 3.26 | 1.01 | 4.66 | 0.35 | 7.24 | 0.70 |
| | CL/F (L/hr/m²) | 2.49 | 1.48 | 3.69 | 0.94 | 3.21 | 0.56 | 2.72 | 1.40 |

What we claim is:

1. A method for treating endometriosis in a mammalian subject comprising contacting the subject with a nucleic acid molecule that modulates the expression of VEGF, VEGFR1, VEGFR2 or combinations thereof under conditions suitable for said treatment.

2. The method of claim 1, wherein said nucleic acid molecule is an enzymatic nucleic acid molecule.

3. The method of claim 1, wherein said nucleic acid molecule is an antisense nucleic acid molecule.

4. The method of claim 1, wherein said nucleic acid molecule is a dsRNA nucleic acid molecule.

5. The method of claim 1, wherein said nucleic acid molecule is a nucleic acid aptamer.

6. The method of claim 1, wherein said nucleic acid molecule comprises a sequence having SEQ ID NO: 13.

7. The method of claim 2, wherein said enzymatic nucleic acid molecule has an endonuclease activity selected from the group consisting of cleavage of RNA encoded by a VEGFRI gene, cleavage of RNA encoded by a VEGFR2 gene and cleavage of RNA encoded by a VEGFR1 gene and RNA encoded by a VEGFR2 gene.

8. The method of claim 2, wherein said enzymatic nucleic acid molecule is in a hammerhead configuration.

9. The method of claim 2, wherein said enzymatic nucleic acid molecule is in an Inozyme configuration.

10. The method of claim 2, wherein said enzymatic nucleic acid molecule is in a Zinzyme configuration.

11. The method of claim 2, wherein said enzymatic nucleic acid molecule is in a DNAzyme configuration.

12. The method of claim 2, wherein said enzymatic nucleic acid molecule is in a G-cleaver configuration.

13. The method of claim 2, wherein said enzymatic nucleic acid molecule is in an Amberzyme configuration.

14. The method of claim 2, wherein said enzymatic nucleic acid molecule is an allozyme.

15. The method of claim 1, wherein said nucleic acid molecule is chemically synthesized.

16. The method of claim 1, wherein said nucleic acid molecule comprises at least one 2′-sugar modification.

17. The method of claim 1, wherein said nucleic acid molecule comprises at least one nucleic acid base modification.

18. The method of claim 1, wherein said nucleic acid molecule comprises at least one phosphate backbone modification.

19. The method of claim 1, wherein said mammalian subject is a human.

20. A method for treating a endometriosis in a mammalian subject comprising administering to the subject a nucleic acid molecule that modulates the expression of VEGF, VEGFR1, VEGFR2 or combinations thereof under conditions suitable for said treatment

21. The method of claim 20 wherein said administration is in the presence of a delivery reagent.

22. The method of claim 21, wherein said delivery reagent is a lipid.

23. The method of claim 22, wherein said lipid is a cationic lipid.

24. The method of claim 22, wherein said lipid is a phospholipid.

25. The method of claim 21, wherein said delivery reagent is a liposome.

26. The method of claim 20, wherein said administration includes treatment with one or more other therapies.

27. The method of claim 26, wherein said other therapies are selected from the group consisting of gonadotropin releasing hormone (GnRH) agonists, Leuprolide Acetate (Lupron Depot), naferalin acetate (Synarel), goserelin acetate (Zolodex), buserelin acetate (Suprefact), Danazol, and oral contraceptives.
